(19) United States
(12) Patent Application Publication
(10) Pub. No.: US 2012/0123468 A1
(43) Pub. Date: May 17, 2012

(54) Wire-Like and Other Devices for Treating Septal Defects and Systems and Methods for Delivering the Same

(76) Inventors: W. Martin Belef, San Jose, CA (US); Dean Carson, Mountain View, CA (US); Ronald J. Jabba, Redwood City, CA (US); Anthony J. Pantages, San Jose, CA (US); Taylor A. Heanue, Oakland, CA (US)

(21) Appl. No.: 13/358,356

(22) Filed: Jan. 25, 2012

Related U.S. Application Data
(63) Continuation of application No. 12/469,466, filed on May 20, 2009, now abandoned.
(60) Provisional application No. 61/054,710, filed on May 20, 2008.

(51) Int. Cl. A61B 17/03

(52) U.S. Cl. 606/213

(57) ABSTRACT

Systems, devices and methods for treating internal tissue defects, such as septal defects, with implantable devices are provided. In some exemplary embodiments, these devices include one or more wires coupled together. The device can include deflectable anchors for engaging the septal tissue.

### CROSS-REFERENCE TO RELATED APPLICATIONS

This application is a continuation of U.S. patent application Ser. No. 12/469,466, filed May 20, 2009, which claims priority to U.S. Provisional Patent Application Ser. No. 61/054,710, filed May 20, 2008, each of which is fully incorporated herein by reference.

### FIELD OF THE INVENTION

The subject matter described herein relates generally to the treatment of septal defects and more particularly, to wire-like implantable devices and systems and methods for their delivery.

### BACKGROUND OF THE INVENTION

During development of a fetus in utero, oxygen is transferred from maternal blood to fetal blood through complex interactions between the developing fetal vasculature and the mother's placenta. During this process, blood is not oxygenated within the fetal lungs. In fact, most of the fetus' circulation is shunted away from the lungs through specialized vessels and foramens that are open during fetal life, but typically will close shortly after birth. Occasionally, however, these foramen fail to close and create hemodynamic problems, which, in extreme cases, can prove fatal. During fetal life, an opening called the foramen ovale allows blood to bypass the lungs and pass directly from the right atrium to the left atrium. Thus, blood that is oxygenated via gas exchange with the placenta may travel through the vena cava into the right atrium, through the foramen ovale into the left atrium, and from there into the left ventricle for delivery to the fetal systemic circulation. After birth, with pulmonary circulation established, the increased left atrial blood flow and pressure causes the functional closure of the foramen ovale and, as the heart continues to develop, this closure allows the foramen ovale to grow completely sealed.

In some cases, however, the foramen ovale fails to close entirely. This condition, known as a PFO, can allow blood to continue to shunt between the right and left atria of the heart throughout the adult life of the individual. A PFO is generally defined herein as an opening existing between two flaps of atrial tissue, the septum primum and the septum secundum.

A PFO can pose serious health risks for the individual, including strokes and migraines. The presence of PFO's have been implicated as a possible contributing factor in the pathogenesis of migraines. Two current hypothesis that link PFO's with migraine include the transit of vasoactive substances or thrombus/emboli from the venous circulation directly into the left atrium without passing through the lungs where they would normally be deactivated or filtered respectively. Other diseases that have been associated with PFO's (and which could benefit from PFO closure) include but are not limited to depression and affective disorders, personality and anxiety disorders, pain, stroke, TIA, dementia, epilepsy, sleep disorders, high altitude pulmonary edema (HAPE), hypoxemia and decompression illness.

To treat PFO's, open heart surgery can be performed to ligate or patch the defect closed. Alternatively, catheter-based procedures have been developed that require introducing umbrella or disc-like devices into the heart. These devices include opposing expandable structures connected by a hub or waist. For instance, with regards to PFO closure, this type of device is generally inserted through the natural PFO opening, or tunnel, with the expandable structures situated on either side of the septum to secure the tissue surrounding the defect between the umbrella or disc-like structure. This type of delivery technique has been referred to as a “through-the-tunnel” technique.

These “through-the-tunnel” devices suffer from numerous shortcomings. For instance, these devices typically involve frame structures that often support membranes, either of which may fail during the life of the patient, thereby introducing the risk that the defect may reopen or that portions of the device could be released within the patient's heart. These devices can fail to form a perfect seal of the PFO, allowing blood to continue to shunt through the defect, especially if the PFO tunnel is excessively long, since these devices have no way to account for significant variations in length. Also, the size and expansive nature of these devices makes safe withdrawal from the patient difficult in instances where withdrawal becomes necessary. The presence of these devices within the heart typically requires the patient to use anti-coagulant drugs for prolonged periods of time, thereby introducing additional health risks to the patient. Furthermore, these devices can come into contact with other portions of the heart tissue and cause undesirable side effects such as an arrhythmia, local tissue damage, and perforation.

In addition to the “through-the-tunnel” technique, closure of the PFO can be accomplished by a “trans-septal” closure technique. In a PFO, the septum primum and septum secundum usually overlap. An implantable device can be inserted through the primum and/or secundum to draw the two flaps of tissue together. This technique is typically referred to as the “trans-septal” closure technique. Devices that are used in trans-septal closure are subject to different design constraints than those that are used in through-the-tunnel techniques. For instance, when the implantable device is delivered through both the primum and secundum, the device can typically be relatively small, but at the same time the device must be strong enough to close the PFO. The device will also experience loads and stress that a through-the-tunnel device would not.

Regardless of the closure technique that is used, there exists a need for implantable devices, and systems and methods for their delivery, for closure of septal defects in the heart.

### SUMMARY

Provided herein are wire-like and other devices configured to treat septal defects, and systems and methods for delivering the same. Although not limited to such, these devices, systems and methods are described in the context of closure of a PFO. The implantable wire-like and other closure devices described herein preferably include anchors for engaging the right and left atrial sides of the septal wall to hold the primum and secundum in proximity with each other to reduce the risk that blood will shunt through the natural PFO tunnel. The configuration of these devices is described in detail by way of the various embodiments, which are exemplary only.

Other systems, methods, features and advantages of the invention will be or will become apparent to one with skill in the art upon examination of the following figures and detailed description. It is intended that all such additional systems, methods, features and advantages be included within this description, be within the scope of the subject matter described herein, and be protected by the accompanying claims. In no way should the features of the exemplary embodiments be construed as limiting the appended claims absent express recitation of those features in the claims.

### BRIEF DESCRIPTION OF THE FIGURES

The details of the invention, both as to its structure and operation, may be gleaned in part by study of the accompanying figures, in which like reference numerals refer to like parts. The components in the figures are not necessarily to scale, emphasis instead being placed upon illustrating the principles of the invention. Moreover, all illustrations are intended to convey concepts, where relative sizes, shapes and other detailed attributes may be illustrated schematically rather than literally or precisely.

FIG. 1A is an exterior/interior view depicting an example human heart.

FIG. 1B is an enlarged side view of the septal wall depicting a PFO taken from the right atrium.

FIG. 1C is an enlarged side view of the septal wall depicting a PFO taken from the left atrium.

FIG. 1D is a cross-sectional view depicting an example PFO region taken along line 1D-1D of FIGS. 1B-C.

FIG. 2A is a side view depicting an exemplary embodiment of an implantable PFO closure device.

FIG. 2B is a top view depicting an exemplary embodiment of an implantable PFO closure device.

FIG. 2C is a perspective view depicting an exemplary embodiment of an implantable PFO closure device.

FIG. 2D is a side view depicting an exemplary embodiment of an implantable PFO closure device.

FIG. 2E is a side view depicting an exemplary embodiment of an implantable PFO closure device within a septal wall.

FIG. 2F is a side view depicting an exemplary embodiment of an implantable PFO closure device.

FIGS. 2G-H are top down views depicting exemplary embodiments of an implantable PFO closure device.

FIG. 3A is a cross-sectional view of an exemplary embodiment of a wire for use with an implantable PFO closure device.

FIG. 3B is a cross-sectional view of an exemplary embodiment of an implantable PFO closure device taken along line 3B-3B of FIG. 2C.

FIG. 3C is a perspective view depicting an exemplary embodiment of wires for use in an implantable PFO closure device.

FIG. 4A is a side view depicting an exemplary embodiment of an implantable PFO closure device.

FIG. 4B is a cross-sectional view of the region 4B in FIG. 4A.

FIG. 4C-F are side views depicting additional exemplary embodiments of an implantable PFO closure device.

FIG. 4G is a cross-sectional view depicting an additional exemplary embodiment of a PFO closure device.

FIGS. 5A-6D are perspective views depicting exemplary embodiments of coupling devices.

FIG. 6E is a cross-sectional view depicting an exemplary embodiment of an implantable PFO closure device.

FIG. 6F is a perspective view depicting an exemplary embodiment a coupling device.

FIG. 6G is an axial cross-sectional view depicting an exemplary embodiment an implantable PFO closure device.

FIG. 6H is a perspective view depicting an exemplary embodiment a coupling device.

FIG. 6I is a radial cross-sectional view depicting an exemplary embodiment of an implantable PFO closure device taken along line 6I-6I of FIG. 6H.

FIGS. 7A-B is a perspective view depicting additional exemplary embodiments of a coupling device.

FIG. 7C is a cross-sectional view depicting an exemplary embodiment of an implantable PFO closure device.

FIG. 8A is a perspective view depicting an exemplary embodiment of a coupling device.

FIGS. 8B-C are perspective views depicting an exemplary embodiment of an implantable PFO closure device.

FIG. 8D is a radial cross-sectional view depicting an exemplary embodiment of an implantable PFO closure device taken along line 8D-8D of FIG. 8C.

FIGS. 9A-E are cross-sectional views depicting exemplary embodiments of an implantable PFO closure device.

FIGS. 9F-G are perspective views depicting exemplary embodiments of a portion of a coupling device.

FIG. 9H is a cross-sectional view depicting an exemplary embodiment of an implantable PFO closure device.

FIG. 9I is a perspective view depicting an exemplary embodiment of an implantable PFO closure device.

FIG. 9J is a cross-sectional view depicting an exemplary embodiment of an implantable PFO closure device taken along line 9J-9J of FIG. 9I.

FIGS. 9K-L are radial cross-sectional views depicting exemplary embodiments of an implantable PFO closure device.

FIG. 10A is a perspective view depicting an exemplary embodiment of an implantable PFO closure device.

FIG. 10B is a radial cross-sectional view depicting an exemplary embodiment of an implantable PFO closure device taken along line 10B-10B of FIG. 10A.

FIGS. 11A-B are side views depicting an exemplary embodiment of an implantable PFO closure device.

FIGS. 11C-D are end views depicting exemplary embodiments of an implantable PFO closure device.

FIG. 11E is a perspective view depicting another exemplary embodiment of an implantable PFO closure device.

FIG. 11F is a side view depicting another exemplary embodiment of an implantable PFO closure device.

FIG. 11G is an enlarged side view depicting region 11G of FIG. 11F.

FIG. 11H is an enlarged side view depicting region 11H of FIG. 11G.

FIGS. 12A-B are perspective views depicting exemplary embodiment of a portion of an implantable PFO closure device.

FIGS. 12C-E are side views depicting an exemplary embodiment of an implantable PFO closure device.

FIG. 12F is a left atrial view depicting an exemplary embodiment of an implantable PFO closure device implanted in a septal wall.

FIG. 13A is a partial cross-sectional view depicting an exemplary embodiment of a delivery system.

FIGS. 13B-C are perspective views depicting exemplary embodiments of portions of a delivery system.

FIGS. 14A-B are side views depicting exemplary embodiments of portions of a delivery system.

### DETAILED DESCRIPTION

Provided herein are implantable septal defect treatment devices and systems and methods for delivering the same. The devices, systems and methods described herein are preferably configured to treat PFOs by the application of an implantable closure apparatus deployable from an intravascular catheter, generally from within an internal lumen of a tissue piercing member or from the external surface of that member. These devices can also be implanted using conventional open heart surgery.

For ease of discussion, these devices, systems and methods will be described with reference to closure of a PFO. However, it should be understood that these devices, systems and methods can be used in treatment of any type of septal defect including ASD's, VSD's and the like, as well as PDA's, pulmonary shunts or other structural cardiac or vascular defects or non-vascular defects, and also any other tissue configuration having overlapping tissue layers including non-defect tissue configurations, non-septal tissue defects and left-atrial appendages (LAA).

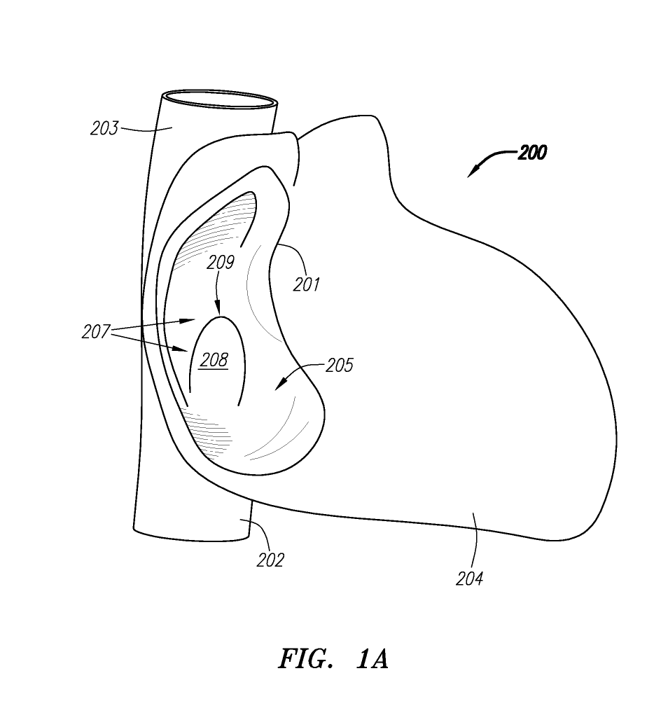

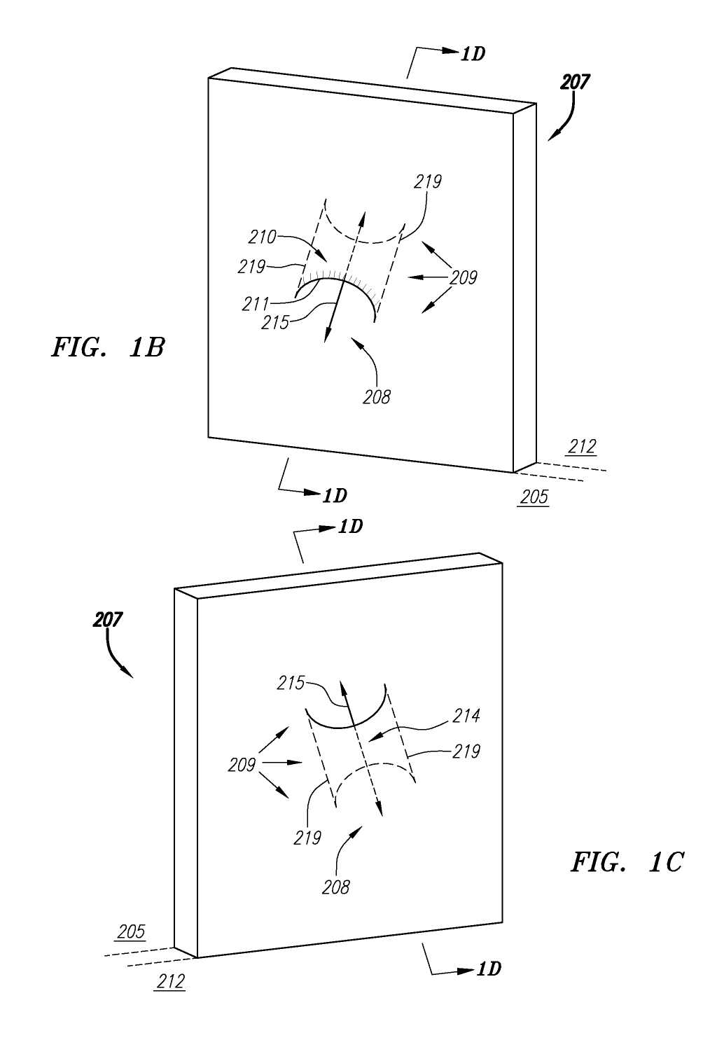

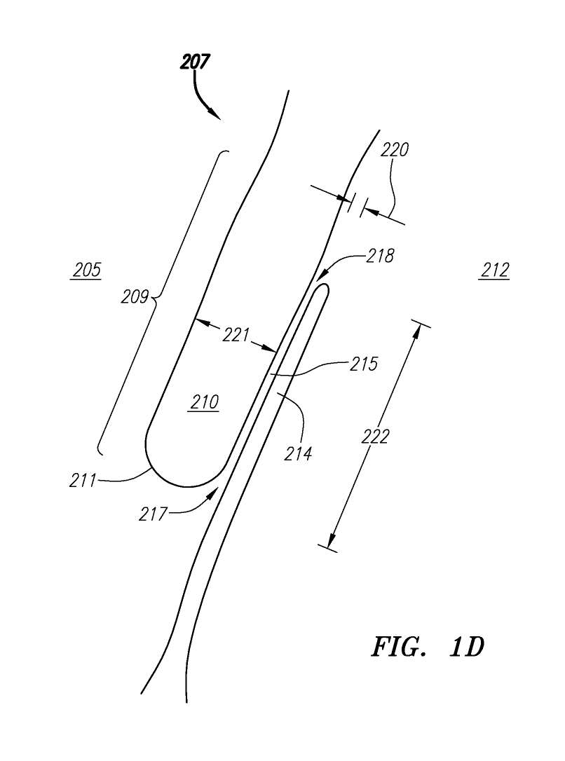

To ease the description of the many alternative embodiments described herein, the anatomical structure of an example human heart having a PFO will be described in brief. FIG. 1A is an exterior/interior view depicting an example human heart 200 with a portion of the IVC 202 and the SVC 203 connected thereto. Outer tissue surface 204 of heart 200 is shown along with the interior of right atrium 205 via cutaway portion 201. Depicted within right atrium 205 is septal wall 207, which is placed between right atrium 205 and the left atrium located on the opposite side (not shown). Also depicted is fossa ovalis 208, which is a region of septal wall 207 having tissue that is relatively thinner than the surrounding tissue. PFO region 209 is located beyond the upper portion of the fossa ovalis 208.

FIG. 1B is an enlarged view of septal wall 207 depicting PFO region 209 in more detail as viewed from right atrium 205. PFO region 209 includes septum secundum 210, which is a first flap-like portion of septal wall 207. The edge of this flap above fossa ovalis 208 is referred to as the limbus 211. FIG. 1C is also an enlarged view of septal wall 207, instead depicting septal wall 207 as viewed from left atrium 212. Here, PFO region 209 is seen to include septum primum 214, which is a second flap-like portion of septal wall 207. Septum primum 214 and septum secundum 210 partially overlap each other and define a tunnel-like opening 215 between sidewalls 219 (indicated as dashed lines in FIGS. 1B-C) that can allow blood to shunt between right atrium 205 and left atrium 212 and is commonly referred to as a PFO.

FIG. 1D is a cross-sectional view depicting an example PFO region 209 taken along line 1D-1D of FIGS. 1B-C. Here, it can be seen that septum secundum 210 is thicker than septum primum 214. Typically, the blood pressure within left atrium 212 is higher than that within right atrium 205 and tunnel 215 remains sealed. However, under some circumstances, conditions can occur when the blood pressure within right atrium 205 becomes higher than the blood pressure within left atrium 212 and blood shunts from right atrium 205 to left atrium 212 (e.g., a valsalva condition).

Many different variations of PFO's can occur. For instance, thickness 220 of septum primum 214, thickness 221 of septum secundum 210, overlap distance 222 and the flexibility and distensibility of both septum primum 214 and septum secundum 210 can all vary. In FIGS. 1B-C, the openings to the PFO tunnel 215 are depicted as being relatively the same size, with the width of tunnel 215, or the distance between sidewalls 219, remaining relatively constant. However, in some cases, one opening can be larger than the other, resulting in a tunnel 215 that converges or diverges as blood passes through. Furthermore, multiple openings can be present, for instance, in the periphery of the primum 214 in the left atrium 212, with one or more individual tunnels 215 extending to the right atrial side. Also, in FIGS. 1B-D, both septum primum 214 and septum secundum 210 are depicted as relatively planar tissue flaps, but in some cases one or both of septum primum 214 and septum secundum 210 can have folded, non-planar, or highly irregular shapes.

For ease of discussion, the devices, systems and methods described herein will be done so with regard to a catheter-based intravascular delivery system routed through the IVC into the right atrium of the heart. A transseptal piercing is performed from the right atrium to the left atrium (“right-to-left”), typically through both the secundum and primum. It should be noted that the devices, systems and methods can also be used when approaching from the SVC into the right atrium, in left-to-right procedures, and in procedures that involve the piercing of either the primum, secundum or both (in either order). These devices, systems and methods can be used in open heart procedures and other minimally invasive procedures as well.

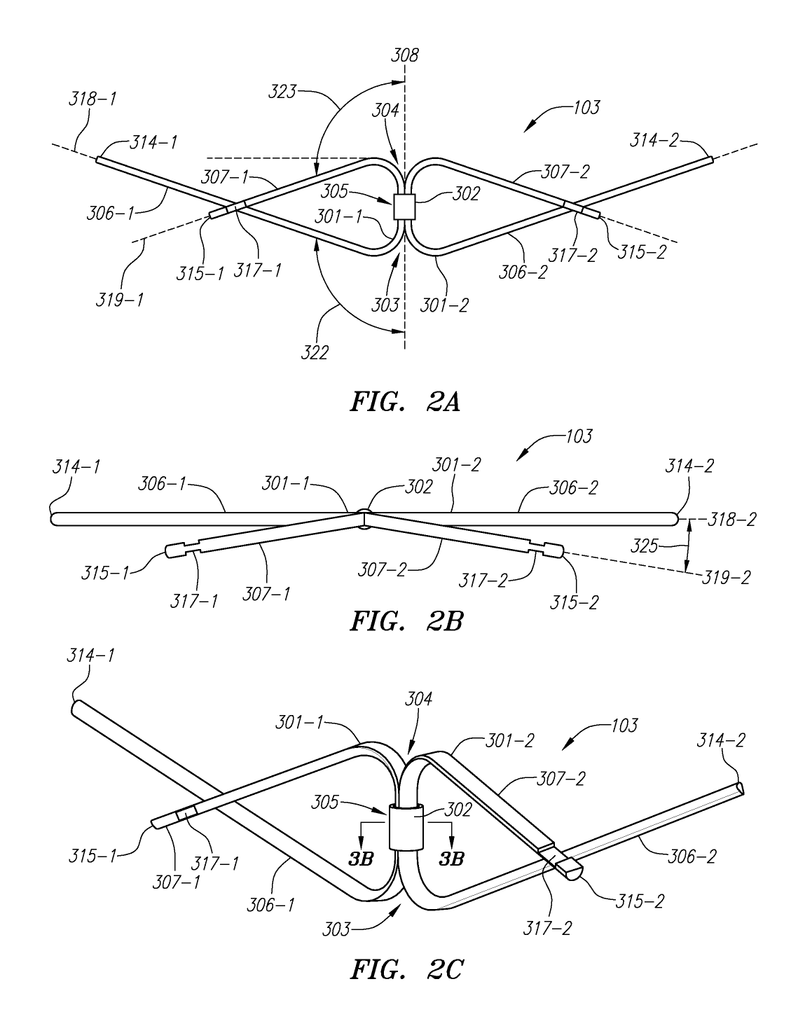

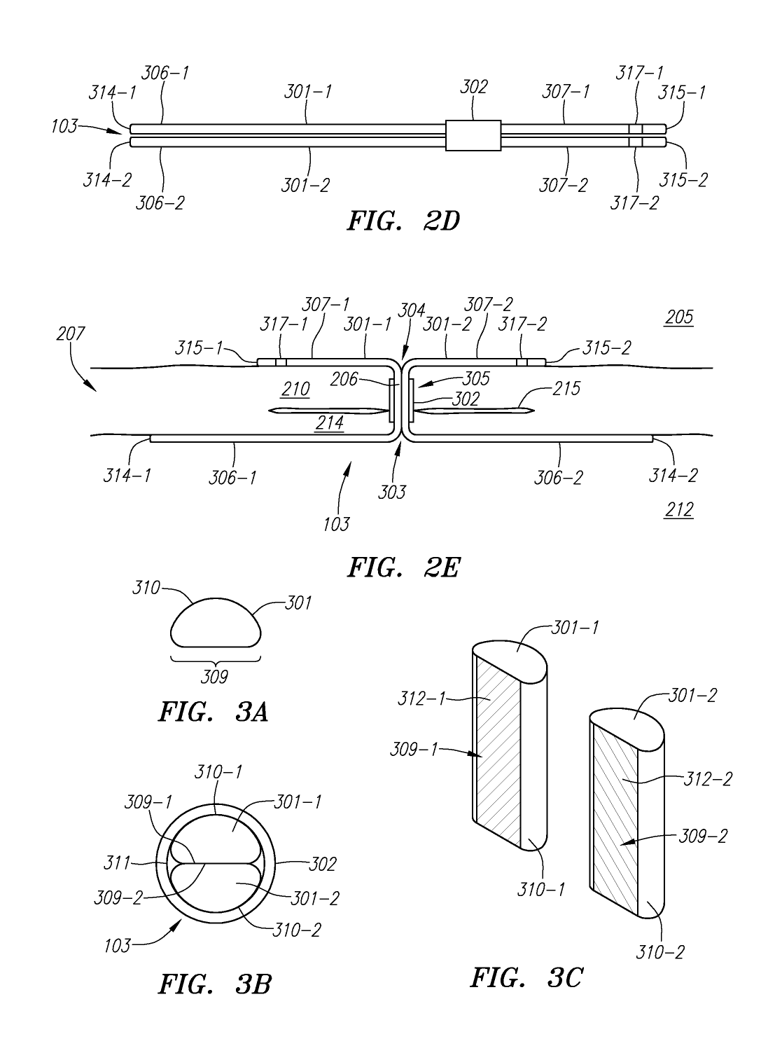

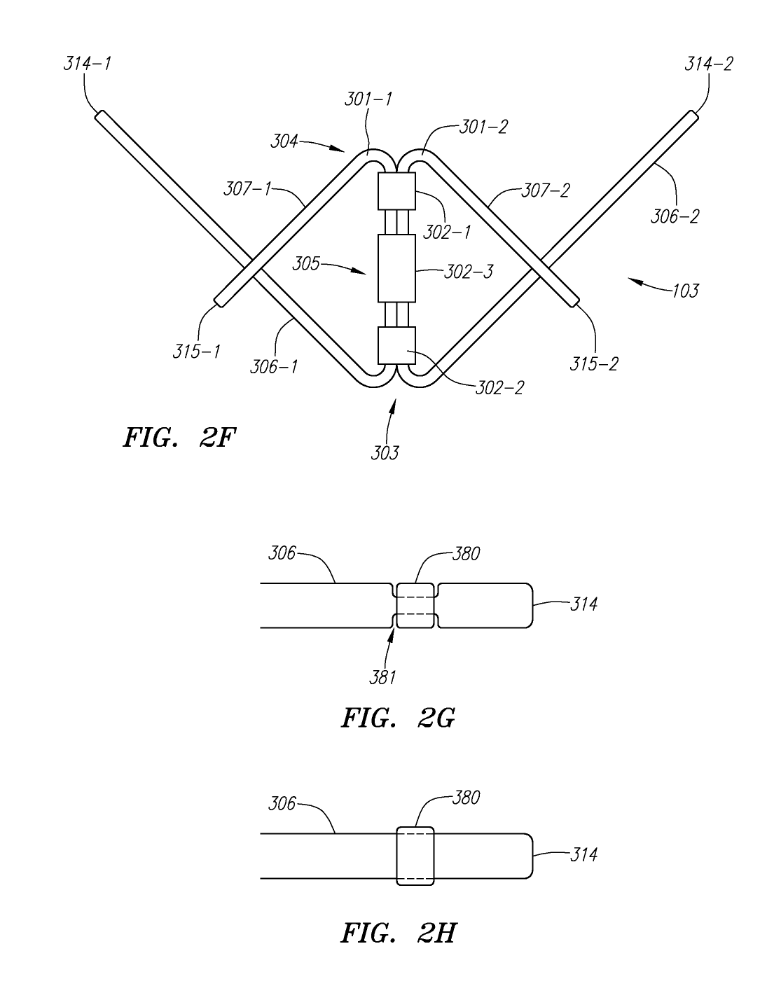

Turning now to the exemplary embodiments, FIG. 2A is a side view depicting an exemplary embodiment of an implantable PFO closure device 103. FIG. 2B is a top view and FIG. 2C is a perspective view of this exemplary embodiment. Implant 103 is configured to close the native PFO tunnel via trans-septal implantation, preferably through both the septum secundum 210 and septum primum 214, as depicted in FIG. 2E. Implant 103 is configured in a clip-like manner, and for ease of discussion herein, will be referred to as clip 103.

Clip 103 preferably includes a left atrial (LA) anchor portion 303, a right atrial (RA) anchor portion 304 and an intermediate, preferably centrally located portion 305. Here, clip 103 includes two deformable wire-like members 301-1 and 301-2 coupled together by way of a coupling device 302. With regards to the reference scheme used herein, generally, specific ones of an element (e.g., wires 301-1 and 301-2) will be referred to using the appendix -#, where the # is a specific one (e.g., 1, 2, 3 . . . N) of the element. When general references are made to the elements, the -# appendix will be omitted. Coupling device 302 is preferably configured to hold wires 301-1 and 301-2 together and maintain their position with respect to each other as well as coupling device 302. The end portions of each wire 301 are deflectable to form septal anchors 306 and 307, which will be referred to as LA members and RA members, respectively. The intermediate portion of each wire 301 between the opposing end portions is generally elongate and straight.

In FIGS. 2A-C, clip 103 is depicted in an exemplary at-rest state. To allow clip 103 to be housed within a delivery device, e.g., a hollow needle and/or catheter, clip 103 is preferably deflectable to a relatively straight, or elongate, configuration (or state) as depicted in the side view of FIG. 2D. Clip 103 is preferably biased to transition from the elongate configuration towards the at-rest configuration depicted in FIGS. 2A-C, although the presence of the septal tissue can prevent clip 103 from fully transitioning to the at-rest state. For instance, as depicted in FIG. 2E, which is a partial cross-sectional view depicting clip 103 implanted within a septal wall having a PFO, the septal tissue holds clip 103 in an intermediate configuration between the at-rest state and the elongate state. Because clip 103 is biased to transition to the at-rest state, LA/RA members 306/307 continue to exert a force on the septal tissue that compresses the tissue therebetween and both helps maintain clip 103 in place within the septal wall 207 and helps maintain the natural PFO tunnel 215 in a closed state.

In FIG. 2E, clip 103 resides within a piercing 206, which is preferably created by the needle in which clip 103 is housed and from which clip 103 is delivered. Exemplary systems and methods for treating septal defects, some of which are configured to enter an off-axis position, as well as supporting devices and methods for facilitating treatment, such as pushers, body members, and proximal controllers and the like, which can be used in conjunction with the devices, systems and methods set forth herein, are described in the following U.S. patent application Publications, each of which are expressly incorporated by reference herein in their entirety: (1) 2006/0052821 entitled “Systems and Methods for Treating Septal Defects”; (2) 2007/0129755 entitled “Clip-Based Systems and Methods for Treating Septal Defects”; (3) 2007/0112358 entitled “Systems And Methods For Treating Septal Defects”; (4) 2008/0015633 entitled “Systems And Methods For Treating Septal Defects,” filed May 4, 2007 and (5) 60/986,229, entitled “Systems, Devices and Methods for Achieving Transverse Orientation in the Treatment of Septal Defects,” filed Nov. 7, 2007. It should be noted, however, clip 103 is not tied to any one specific method of implantation, and can be used with any desired PFO closure technique or with any desired PFO closure delivery system.

When implanted in the septal wall, as depicted in FIG. 2E, LA portion 303 is preferably located in left atrium 212 and RA portion 304 is preferably located in the right atrium 205. LA members 306 are preferably relatively longer than RA members 307 to apply a closure force to a relatively wider region of septal tissue. It should be noted that the respective lengths of LA members 306 and RA members 307 can be the same or can vary. If desired, RA members 307 can be relatively longer than LA members 306 and/or each LA member 306 (or RA member 307) can have a different length, etc.

In this embodiment, the number of LA/RA members 306/307 can be varied depending on the number of wires 301 that are used. If only one LA member 306 and RA member 307 is desired, then only one wire 301 can be used. In such an embodiment, coupling device 302 can be omitted. It should be noted that the number of LA/RA members 306/307 can also be varied by coupling additional members to each wire 301 or by further splitting each wire 301. If multiple wires 301 are used, the cross-sectional profile of those wires 301 can be configured to complement each other, such that a gap does not exist along the center axis of the device. As will be discussed in more detail below, the two wires 301 preferably have a “D” shaped cross-section. If three or more wires 301 are used, the cross-sectional profile of each wire can have a generally circular sector shape (i.e., a pie slice-shape). LA/RA members 306/307 can be configured, arranged and oriented with respect to each other in numerous different ways, including those described in the incorporated '358 publication.

Referring back to FIGS. 2A-B, LA members 306 each have a longitudinal axis 318 and an end tip 314. RA members 307 also have a longitudinal axis 319 and an end tip 315. End tips 314 and 315 are preferably configured to be atraumatic and substantially dull. RA members 307 also include neck regions 317, located on the end portion near each end tip 315. The use and function of neck regions 317 will be described in further detail below.

Clip 103 has a longitudinal axis 308 and the degree of deflection of LA members 306 and RA members 307 from longitudinal axis 308 is referred to herein as LA deflection 322 and RA deflection 323, respectively. As shown here, LA deflection 322 and RA deflection 323 both exceed 90 degrees. The actual LA deflection 322 that is implemented can be dependent on at least two factors. A larger deflection typically results in the ability to apply a greater compressive force but at the same time, the force can cause the clip to rotate or to move too far in a distal direction during deployment, which can interfere with the proper placement of RA members 307. For example, if the deployment of LA members 306 causes clip 103 to move too far distally before RA members 307 are deployed, then RA members 307 could be drawn into and trapped partially within the actual tissue piercing, preventing the desired amount of deflection. Preferably, LA deflection 322 and RA deflection 323 are both between 90 and 135 degrees and, most preferably between 95 and 100 degrees.

As shown here, RA members 307 can be offset from LA members 306. The respective offset between the longitudinal axis 319 of RA member 307 and the longitudinal axis 318 of LA member 306 is depicted in FIG. 2B as offset angle 325. Here, offset angle 325 is approximately 15 degrees. The offset of RA members 307 with respect to LA members 306, among other advantages, allows RA members 307 and LA members 306 to deflect past each other such that the members cross or overlap as depicted in FIG. 2A. Whether two adjacent LA/RA members 306/307 actually overlap in the at-rest state is, of course, dependent on the degree of deflection and the length of each LA/RA member 306/307.

In this embodiment, RA members 307 are deflected towards each other as depicted in FIG. 2B, whereas LA members 306 remain directly in line with each other. It should be noted that, of course, LA members 306 can also be deflected towards each other with RA members 307 remaining directly in line with each other or, both RA members 307 and LA members 306 can be deflected towards their respective counterpart. It should also be noted that the offset angle 325 can be any desired angle and is not limited to 15 degrees. In a preferred embodiment, offset angle 325 is minimized so that RA members 307 can overlap with LA members 306, but RA members 307 still extend away from each other to maximize the amount of septal tissue that is engaged by the RA members 307. As depicted in FIG. 2B, both RA members 307 are offset beneath LA members 306; however, it should be noted that in other embodiments, RA member 307-1 can be offset above or beneath LA member 306-1, while RA member 307-2 is offset to the opposite side as RA member 307-1.

Wires 301 are preferably formed from a biocompatible material, which can be either elastic (e.g., stainless steel, various polymers, elgiloy and the like) or superelastic (e.g., nickel-titanium alloys such as nitinol, Chrome-doped nitinol and the like). Wires 301 can be formed from wire stock or can be separated from sheet stock material by use of machine or laser cutting tools, electrical discharge machining (EDM), chemical etching and the like. In a preferred embodiment, wires 301 are formed from nitinol wire stock and heat treated to retain the at-rest state depicted in FIGS. 2A-C.

Wires 301 can be formed from nitinol wire stock in a D-shape or other configuration by any desired method, such as roll milling, coining and the like. Roll milling of circular wire stock is a progressive process where wire is drawn axially through a set of rotating rigid cylinders. Coining, a closed die squeezing process, can be used to form segments of circular drawn wire into cross sections of the D-shape or any other desired geometry. The wire is confined between two contoured dies that close along rigid guides that are perpendicular to the axis of the wire.

Coupling device 302 is preferably formed from a biocompatible material such as nitinol, elgiloy, stainless steel, polymeric materials and the like. When in a tubular shape, coupling device 302 is preferably formed by cutting or machining a section from tube stock. Alternatively, coupling device 302 can be molded in the cylindrical or other desired shape, or can be fabricated from ribbon, wire or sheet material and then manipulated to assume the desired shape. The free edges can then be sealed together by welding, soldering, the use of adhesive and the like. It should be noted that, although coupling device 302 is described as being generally cylindrical in many of the embodiments herein, any shape can be used as desired. Although not required, coupling device is preferably shaped in a manner similar to the profile of the wires 301, unless otherwise noted herein.

Although many embodiments are described herein as having a single coupling device 302, it should be noted that any number of coupling devices 302, having the same size and/or configuration can be used and placed in any desired manner. FIG. 2F is a side view depicting an exemplary embodiment of clip 103 having three coupling devices 302-1 and 302-2 placed directly adjacent to RA members 307 and LA members 306, respectively, and a relatively larger coupling device 302-3 is placed in the center of portion 305. Although shown in spaced relation to each other here, the adjacent coupling devices can also abut each other to provide increased resistance to slippage.

To facilitate external imaging by the user, clip 103 can be configured with markers such as radiopaque marker on any portion thereof. FIGS. 2G-H are top down views depicting end portions of exemplary embodiments of an LA member 306 having a radiopaque (RO) marker 380. LA member 306 can have a recessed portion 381 (indicated in part by dashed line) on which tubular RO marker 380 can be coupled as depicted in FIG. 2G. Here, RO marker 380 lies generally flush with the edge of LA member 306. Alternatively, RO marker 380 can be coupled directly to LA member 306 on a non-recessed portion (indicated by dashed line). RO marker 380 can be coupled in any desired fashion, such as by crimping, adhesives, welding, soldering, thermal or cryogenic adjustment and the like.

FIG. 3A is a cross-sectional view of a wire 301 suitable for use with the implantable clip. Here, wire 301 has a D-shape with a relatively flat, or planar, surface 309 located next to a curved surface 310. FIG. 3B is a cross-sectional view of clip 103 showing clip 103 along line 3B-3B of FIG. 2C (for ease of illustration, LA members 306 are not shown). Here, wires 301-1 and 301-2 are shown held together by coupling device 302, which preferably locks wires 301-1 and 301-2 together in fixed relation to each other as well as to coupling device 302 itself. To facilitate this, wires 301-1 and 301-2 can be optionally joined with adhesive, welded or soldered together to more securely lock them together. Although shown here with a circular peripheral profile, coupling device 302 and a portion of the inner surface of the delivery device can have matching, non-circular profiles that allow clip 103 to maintain a particular orientation within the delivery device (e.g., circular, elliptical, polygonal, asymmetric or irregular profiles).

Furthermore, any portion of planar surfaces 309-1 and 309-2 can be textured to increase the surface friction between them and thereby increases the amount of force necessary to remove either wire 301 from coupling device 302. FIG. 3C is a perspective view depicting an exemplary embodiment of wires 301-1 and 301-2. Here, sections of wires 301-1 and 301-2 are shown having a texture on planar surfaces 309-1 and 309-2, respectively. Any portion of curved wire surfaces 310-1 and 310-2 and/or the inner surface of the coupling device can also be textured to increase the surface friction between them.

In this embodiment, the textured surface includes a plurality of grooves 312 that are oriented in complementary fashion such that they tend to interlock with the corresponding grooves on the other wire when joined together. One of skill in the art will readily recognize that many different types of surface textures can be applied and, accordingly, the present subject matter is not limited to any one surface texture.

Numerous different techniques can be used to attach coupling device 302 to wires 301 such that the wires 301 and coupling device 302 remain locked into place and fixed with respect to each other. Although minimal movement could occur in some applications, preferably the wires 301 and coupling device 302 remain locked to maximize the stability of clip 103 while located within the septal wall. The following embodiments describe various techniques for attachment of coupling device 302 to wires 301. As mentioned already, attachment methods such as those involving adhesives, welding (e.g., laser and thermal), soldering and the like can each be used.

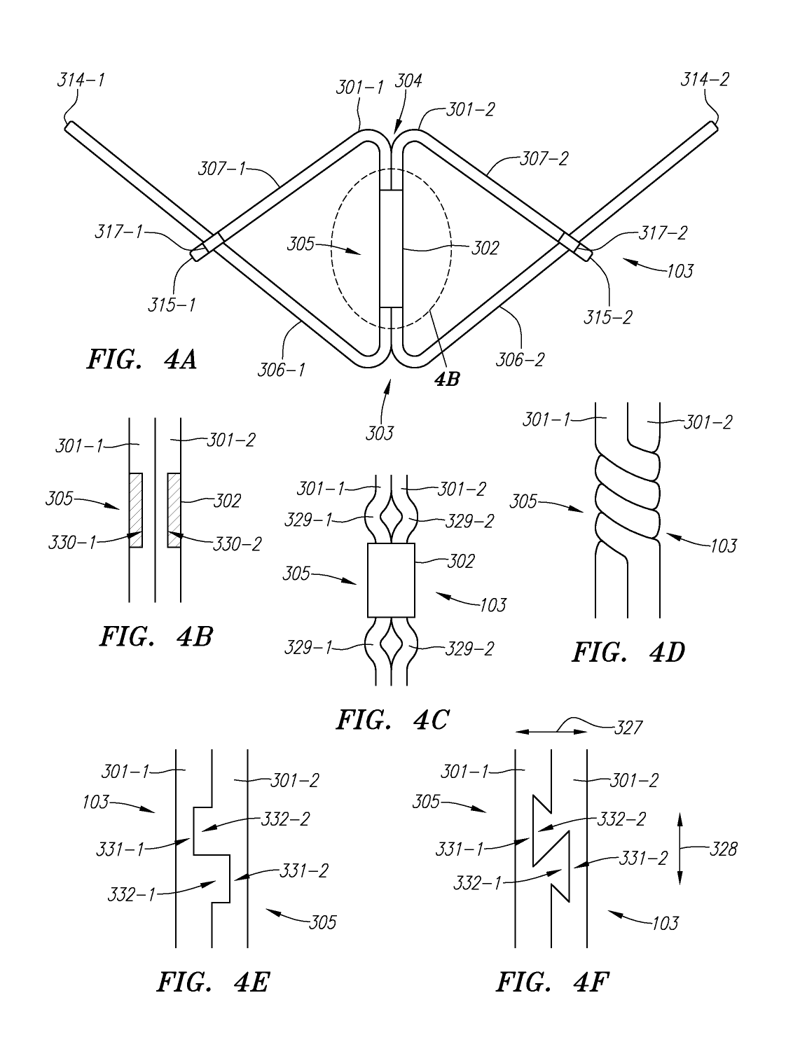

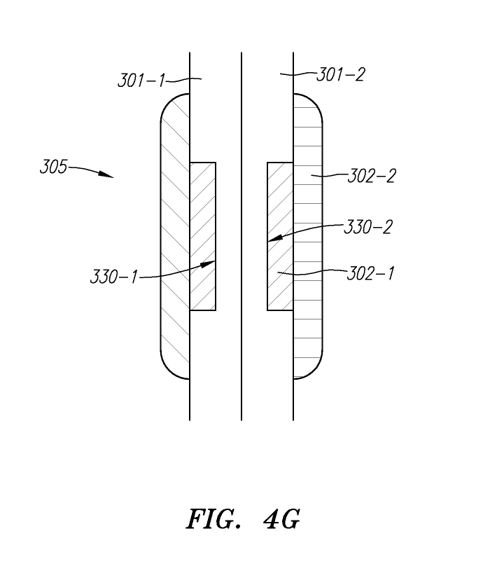

Although FIGS. 3A-C depict an exemplary embodiment of clip 103 having wires 301 with generally D-shaped cross-sectional profiles, it should be noted that wires 301 can have any cross-sectional profile, or combination of cross-sectional profiles, desired for the particular application. Circular, elliptical, polygonal, irregular, asymmetrical, annular, hollow, and the like are all examples of profiles that can be used. In the instance where a profile is used that results in less surface area contact with the coupling device, such as elliptically profiled wires in a generally circular coupling device, additional techniques can be used to increase the locking potential. For instance, an adhesive can be used to fill any gaps or free space between the wires and the coupling device, and between the wires themselves. Multiple, overlapping coupling devices can be used, such as will be described with respect to FIG. 4F. Multiple coupling devices placed end-to-end, similar to that described with respect to FIG. 2F, can also be used. In addition, other types of wire such as braided wire can be used and other non-elongate wire configurations, such as coiled or wound and the like, can be used. As mentioned, various combinations of differing cross-sectional profiles, wire types and/or configurations can be used. For instance, in one exemplary embodiment, wires 301 have D-shaped profiles in the central portion where the coupling device is placed, and transition to circular profiles in the proximal and distal portions (e.g., the portions having arm members 306/307). In another exemplary embodiment, wires 301 have, for example, a solid wire core with a circular profile and a braided wire outer core. In still another exemplary embodiment, wires 301 have a generally D-shaped profile and transition to braided wire or coiled wire tips at the ends of any of the LA and/or RA members 306/307.

FIG. 4A is a side view depicting another exemplary embodiment of clip 103 and FIG. 4B is a cross-sectional view of the region 4B in FIG. 4A. In this embodiment, clip 103 is configured such that coupling device 302 resides generally flush against wires 301. One or both of wires 301 can include a recessed portion configured to receive the coupling device. As seen in FIG. 4B, each wire 301 includes recessed portion 330, which allows the reduction of the cross-sectional profile of clip 103. This provides a more stable interface between wires 301 and coupling device 302, reducing the risk that coupling device 302 will slide out of position. This reduced profile can also allow a smaller needle/catheter to be used in delivering clip 103, which in turn can allow the needle/catheter to be more flexible, thereby facilitating navigation through the patient's vasculature. The resulting smaller puncture in the patient's septal wall minimizes residual bleeding, both around and through the puncture, which improves the healing time.

Although coupling device 302 is shown to reside in a flush configuration against the exterior surface of wires 301, any reduction in overall profile of clip 103 will provide the aforementioned benefits to some degree. FIG. 4C is a side view depicting central portion 305 of another exemplary embodiment of clip 103. Here, coupling device 302 is positioned between raised portions 329 on the generally straight portions of each wire 301. Although this embodiment does not substantially reduce the clip profile, it can provide a more stable interface between coupling device 302 and wires 301. Unless otherwise noted, configuration of clip 103 in the manner described with respect to FIGS. 4A-C can be applied with any embodiment described herein.

Wires 301-1 and 301-2 can also be configured to lock with respect to each other independent of coupling device 302. For instance, FIG. 4D is a side view of central portion 305 of an exemplary embodiment of clip 103 where wires 301-1 and 301-2 are twisted. Twisting the wires 301 can lock them into place with respect to each other. Coupling device 302 (not shown) can then be optionally applied over wires 301-1 and 301-2 for added stability and strength.

FIG. 4E is a similar view of another exemplary embodiment where wires 301-1 and 301-2 have complementary features that interlock together. Here, wire 301-1 includes a slot feature 331-1 and a tab feature 332-1 which are configured to interface with the complementary features 331-2 and 332-2, respectively, on wire 301-2. These features 331 and 332 provide act to resist slippage between wires 301-1 and 301-2. It should be noted that any number of one or more complementary pairs of features can be used (two are shown here). Similar to the embodiment described with respect to FIG. 4D, this embodiment is preferably implemented with coupling device 302 (not shown). It should also be noted that complementary features can be used between wires 301 and coupling device 302. For instance, one or both of wires 301-1 and 301-2 can have a slot in which a complementary tab located on the coupling device can be inserted.

FIG. 4F depicts another embodiment similar to FIG. 4E where features 331 and 332 are further configured to provide relatively more secure interlocking capacity. In this embodiment, wires 301-1 and 301-2 will resist being pulled apart in direction 327 in addition to resisting slippage in the vertical direction 328. As with the embodiments described with respect to FIGS. 4A-C, the embodiments described with respect to FIGS. 4D-F, unless otherwise noted, can be implemented with any embodiment described herein.

Referring back to FIGS. 4A-B, there are various ways in which coupling device 302 can be securely fit within recessed portion 330 of wires 301. For instance, in one exemplary embodiment, coupling device 302 and/or wires 301 can be cryogenically manipulated to allow coupling device 302 to change in diameter. For instance, in one exemplary embodiment, coupling device 302 is formed from a temperature responsive material such as nitinol. Coupling device 302 can first be sized to the appropriate internal diameter by cooling device 302 to a low temperature, such as −40 degrees Celsius (−40 C), such that device 302 expands and can be placed over a sizing mandrel having the preferred outside diameter. Once the coupling device is positioned on the sizing mandrel, the assembly can then be heat treated at a much higher temperature, such as 520 C, to instill the preferred internal diameter. After heat treatment, coupling device 302 can be chilled and then removed from the sizing mandrel.

In order to advance the coupling device 302 onto the wires 301, coupling device 302 is first chilled to expand device 302. Wires 301 can then be advanced through coupling device 302. Wires 301 are joined by coupling device 302 and then returned to room temperature. During the return to room temperature, coupling device 302 shrinks, locking onto wires 301. Clip 103 can then undergo additional heat treatments as needed (e.g., to instill a bias for members 306/307 to deflect). Placement of coupling device 302 within the recessed portions 330 of wires 301 also facilitates the placement of a second coupling device over the first. For instance, FIG. 4G is a cross-sectional view, similar to FIG. 4B, showing center section 305 of clip 103 having a first coupling device 302-1 locked within recessed portions 330, and a second coupling device 302-2 locked in place over coupling device 302-1. Such a configuration can provide added resistance to wire slippage.

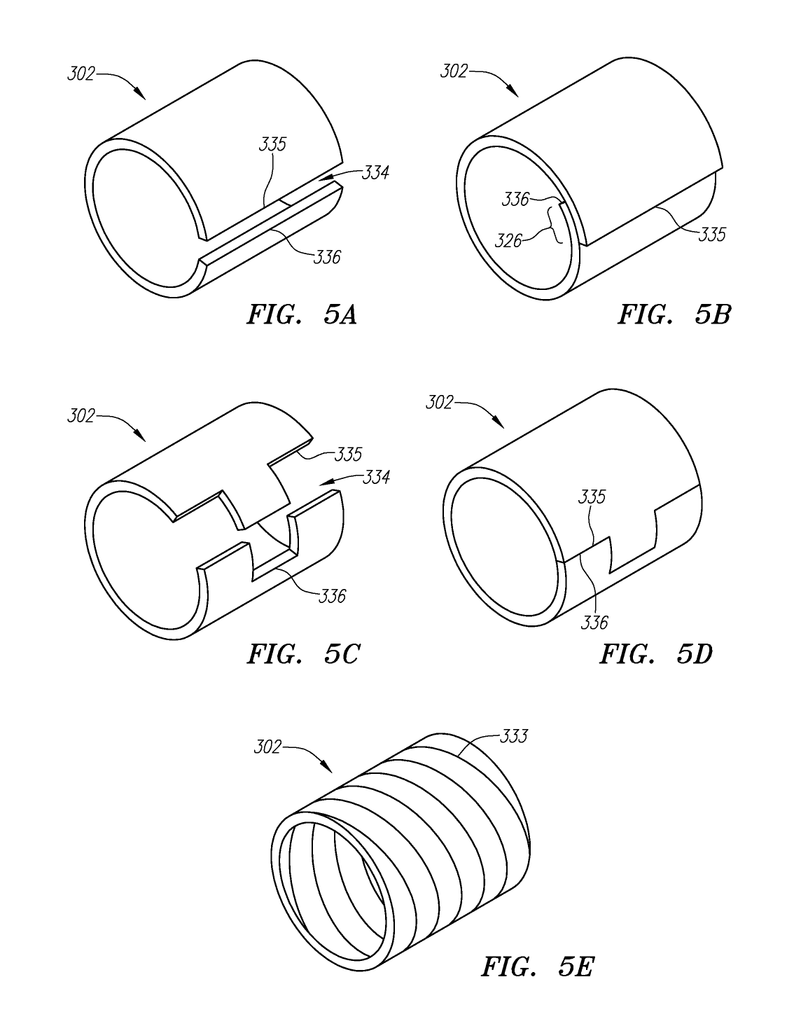

FIGS. 5A-E are perspective views depicting exemplary embodiments of coupling device 302 having the capability to transition from a relatively expanded state to a reduced, or compressed state. The expanded state is preferably large enough to allow device 302 to be advanced over wires 301 (not shown) and into the desired position. Once in position, device 302 is preferably placed into the smaller compressed state to lock the components of clip 103 (not shown it its entirety) together. Transition between the two states can be accomplished in a variety of ways. For instance, coupling device 302 can be fabricated in either the expanded state, the compressed state or some intermediate state and simply mechanically deformed to the desired state.

Alternatively, coupling device 302 can be formed from a nickel-titanium alloy (e.g., nitinol) or other shape retentive material and can be heat treated in the compressed state so as to be mechanically biased towards that configuration. Coupling device 302 can then be expanded from the compressed configuration while fitting it over wires 301. Once into position, coupling device 302 can be released to return to the compressed state and thereby lock the components of clip 103 together.

FIG. 5A depicts an exemplary embodiment of coupling device 302 having longitudinal free edges 335 and 336 separated by longitudinal opening 334. Coupling device 302 can be slid over wires 301 (not shown) in this configuration and then compressed to decrease the inner diameter of coupling device 302 and securely lock coupling device 302 into place over wires 301 (not shown). Although coupling device 302 can be compressed such that edges 335 and 336 are in direct contact, FIG. 5B depicts an alternative embodiment where coupling device 302 is compressed with a region of overlap 326 between the opposing edges 335 and 336.

FIG. 5C is a perspective view depicting another exemplary embodiment of coupling device 302 similar to that described with respect to FIGS. 5A-B. Here, instead of having a generally straight longitudinal opening 334, a stepped shape is formed in the opposing edges 335 and 336 to provide an interlocking capability when compressed with edges 335 and 336 in proximity with each other, as shown in FIG. 5D. This interlocking capability provides further stability to coupling device 302 when in the compressed state.

FIG. 5E is a perspective view depicting another exemplary embodiment of coupling device 302. Here, device 302 is configured as a tubular coil. A continuous slot 333 is present about the circumference of device 302, allowing the device to expand from the compressed state shown here. Preferably, device 302 is biased towards this compressed state. It should be noted that based on the description herein, one of skill in the art will recognize that a myriad of other coil-like devices can be used for coupling device 302, not limited to the tubular configuration described with respect to FIG. 5E. For instance, helical and other coils wound from wire or ribbon-like materials could also be used.

When implementing embodiments the same as or similar to those described with respect to FIGS. 5A-E, it should be noted that, if the free edges are in contact with each other or if there is an overlapping contact region, when in the compressed state, then the coupling device can be secured in the compressed state by coupling the free edges (or overlapping region) together using any desired attachment technique, including but not limited to the use of adhesives, soldering, laser or thermal welding, and the like.

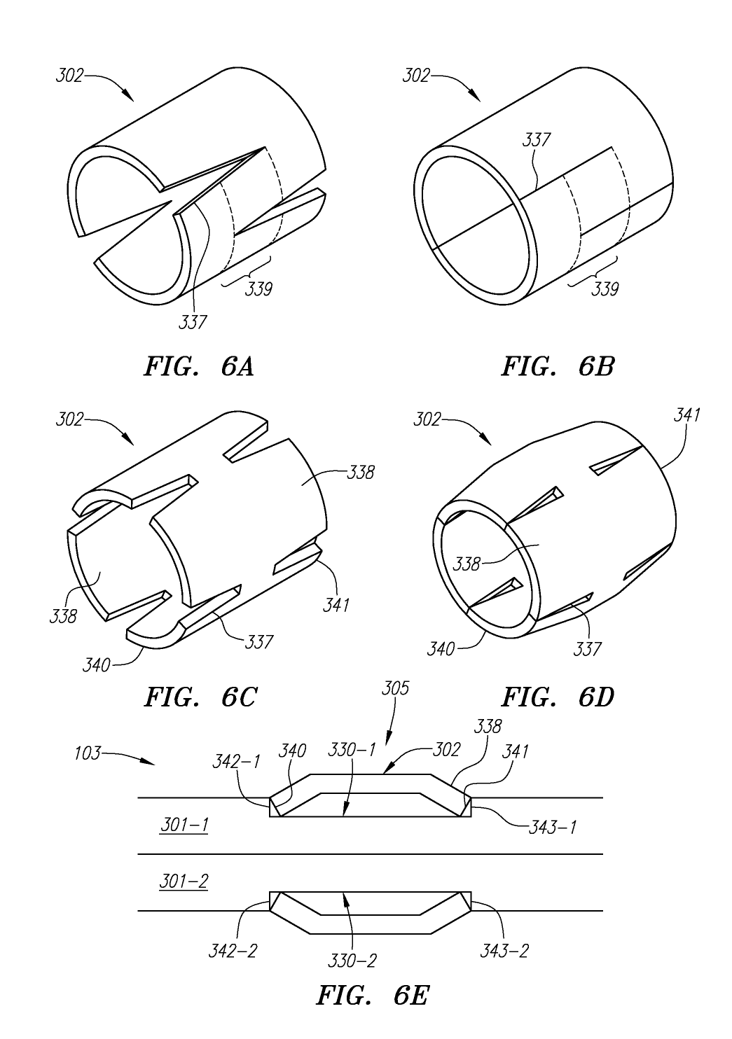

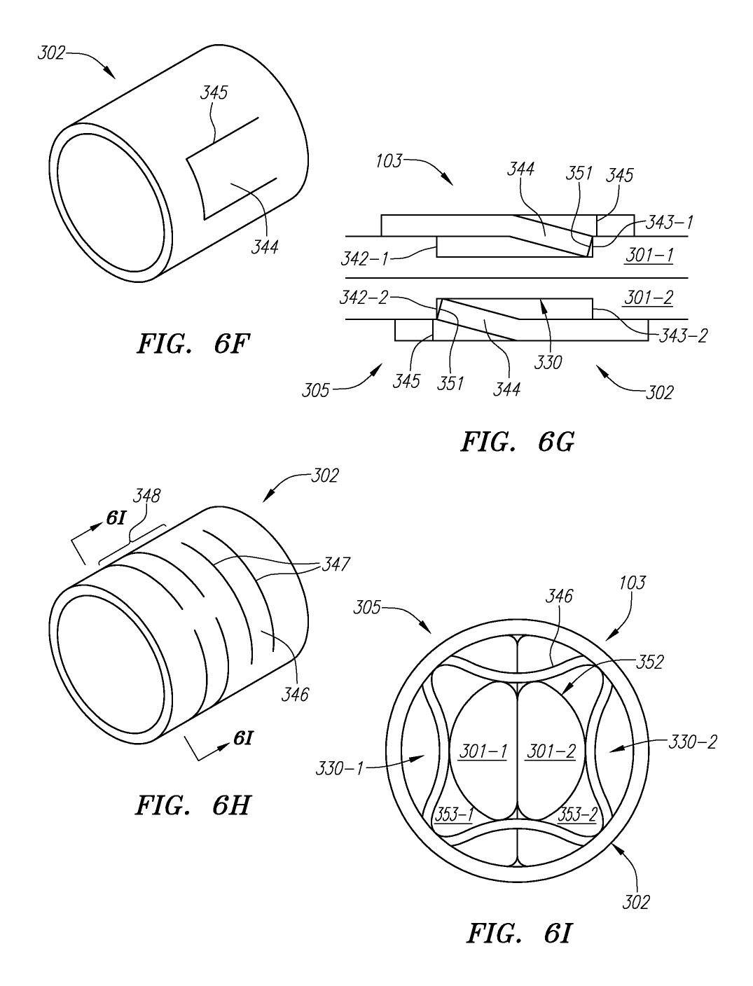

FIGS. 6A-B are perspective views depicting another exemplary embodiment of coupling device 302. Here, coupling device 302 has multiple overlapping slots 337 which can be opened to expand the diameter of coupling device 302. For instance, FIG. 6A depicts coupling device 302 with slots 337 expanded, while FIG. 6B depicts coupling device 302 with slots 337 in a relatively less open, compressed state having a smaller diameter. Slots 337 preferably overlap in region 339 to allow the overall diameter of coupling device 302 to be changed. A greater overlap between slots 337 will correspond to a greater ability to change the diameter of coupling device 302.

FIGS. 6C-D are perspective views depicting another exemplary embodiment of coupling device 302, in the expanded and compressed states, respectively. Here, each end of coupling device 302 includes multiple slot openings 337, which have a generally triangular or tapered shape. The portions between adjacent slots 337 form tabs 338. The device depicted in FIG. 6C can be compressed into the configuration depicted in FIG. 6D where the gaps within slots 337 have been reduced and tabs 338 are deflected toward each other. This reduces the overall diameter of coupling device 302 on either end. The end edges 340 and 341 of coupling device 302 preferably contact abutments located on wires 301 (not shown).

FIG. 6E is a cross-sectional view depicting clip 103 having this embodiment of coupling device 302 placed thereon. Here, wires 301 each have a recessed portion 330 and the edges 342 and 343 form the abutments that contact edges 340 and 341, respectively, of coupling device 302. Alternatively, raised portions can be formed on wires 301 to act as the abutments. This configuration allows coupling device 302 to be advanced over wires 301 in the expanded, or non-deflected state until in position at which point tabs 338 can be deflected inwards to engage with the abutments on wires 301 and thereby lock the components of clip 103 together. It should be noted that any shape slots 337 and tabs 338 can be used so long as they allow the ends of coupling device 302 to compress over wires 301.

When implementing embodiments the same as or similar to those described with respect to FIGS. 6A-E, it should be noted that, if the edges of the slots are in contact with each other when in the compressed state, then the coupling device can be secured in the compressed state by coupling those edges together using any desired attachment technique, including but not limited to the use of adhesives, soldering, laser or thermal welding, and the like.

FIGS. 6F-I depict additional exemplary embodiments of coupling device 302 having deflectable tabs. FIGS. 6F-G are a perspective view and an axial cross-sectional view, respectively, of coupling device 302 having a slot 345 placed in opposite sides of the tubular body. The presence of slot 345 creates a deflectable tab 344 as shown here. FIG. 6G depicts coupling device 302 locked into place over wires 301 (wires 301 are not shown in FIG. 6F). In this embodiment, wires 301 each include a recessed portion 330 having end edges 342 and 343. Preferably, tabs 344 on coupling device 302 deflect inwards into the recessed portions 330 such that edge 351 of tab 344 contacts one of the edges of recessed portion 330, either edge 342 or 343, depending on the orientation of tab 344. Tabs 344 are oriented opposite to each other as shown so that coupling device 302 is locked into place and will resist movement in either direction along wires 301. It should be noted that any number of one or more tabs 344 can be used with this embodiment.

FIG. 6H is a perspective view of another exemplary embodiment of coupling device 302. Here, coupling device 302 includes multiple pairs of slots 347 arranged to create deflectable tabs 346. FIG. 6I is a radial cross-sectional view of coupling device 302 taken along line 6I-6I of FIG. 6H and also showing the presence of wires 301 therein (wires 301 are not shown in FIG. 6H). Here, it can be seen that each tab 346 preferably deflects into recessed portion(s) 330 of wires 301. Tabs 346 can be configured to deflect and contact both the base surface 352 and the end surfaces 353 of recessed portion 330 of wires 301 or can deflect partially into recessed portion 330, contacting only end surfaces 353.

Referring back to FIG. 6H, tabs 346 are preferably spaced along region 348, which preferably has the same length as the length of any corresponding recessed portions along the longitudinal axis (e.g., center axis 308, which is not shown) of the implantable clip. This provides a stable fit for coupling device 302 over the wires and prevents coupling device 302 from sliding. Tabs 346 are shown as being connected on both sides, i.e., tabs 346 have two unconnected free edges located opposite each other, but it should be noted that a continuous “U” shaped slot can be formed so as to give tabs 346 a configuration similar to that of FIG. 6F. It should be noted that any number of one or more tabs 346 can be used with this embodiment.

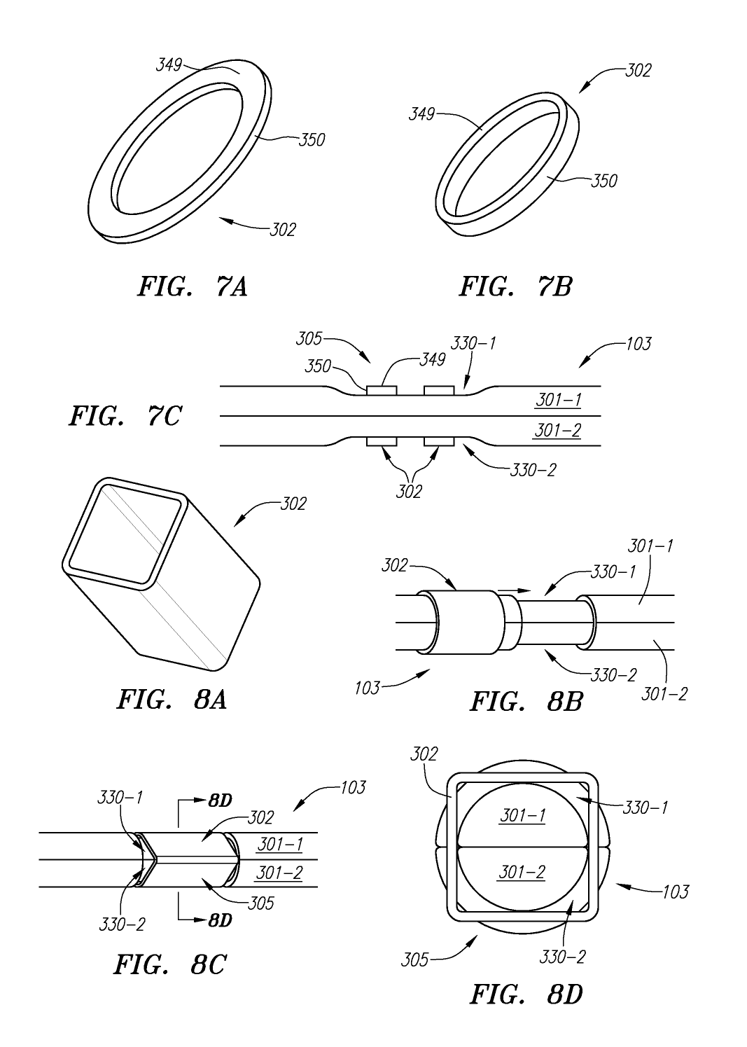

FIG. 7A is a perspective view depicting another exemplary embodiment of coupling device 302. Here, coupling device 302 has an annular, ring-like, configuration with a top edge denoted as surface 349 and an outer edge denoted as surface 350. The configuration depicted in FIG. 7A is preferably formed from a sheet of material having elastic or superelastic properties. The configuration depicted in FIG. 7A can be modified, or inverted, to that of the perspective view of FIG. 7B. Here, it can be seen that surface 350 has become the top surface and surface 349 has become the inner surface of coupling device 302.

FIG. 7C depicts several of these coupling devices 302 located within recessed portions 330 of wires 301. To place coupling devices 302 on wires 301, the device is preferably advanced over wires 301 when in the configuration of FIG. 7A. When in the desired position, coupling devices 302 can be inverted into the configuration shown in FIGS. 7B-C. This inverted configuration has a relatively smaller inner diameter that causes coupling device 302 to lock onto the surface of wires 301.

FIG. 8A is a perspective view of another exemplary embodiment of coupling device 302. Here, coupling device 302 has a hollow, box-like shape with a generally square cross-sectional profile. The configuration depicted in FIG. 8A can be deformed from this at-rest, compressed state to another, expanded state having a relatively larger inner diameter, or width.

For instance, FIG. 8B is a perspective view showing coupling device 302 while being advanced over wires 301. Here, coupling device 302 has been deformed from the generally box-like configuration to a generally cylindrical configuration with a larger inner diameter that allows coupling device 302 to be advanced over wires 301. Once over recessed portion 330, coupling device 302 is allowed to revert (or is reverted) to or towards its box-like configuration as depicted in FIG. 8C. In some embodiments, it can be desirable for coupling device 302 to revert to an intermediate state between the box-like and cylindrical configurations, where a continuous compressive force is applied to wires 301. It should be noted that coupling device 302 does not need to convert between either a fully square/rectangular configuration or a fully cylindrical (having a circular cross-section) configuration, since some residual deformity from each configuration can persist after transformation.

FIG. 8D is a cross-sectional view of clip 103 taken along line 8D-8D of FIG. 8C. Here, it can be seen that coupling device 302 has the generally square cross-sectional profile. Coupling device 302 can be configured with other cross-sectional profiles for the at-rest configuration. For instance, instead of a generally square profile, a generally triangular profile or a generally elliptical profile could be used. One of skill in the art will readily recognize the many different profiles that can be used in light of the description herein.

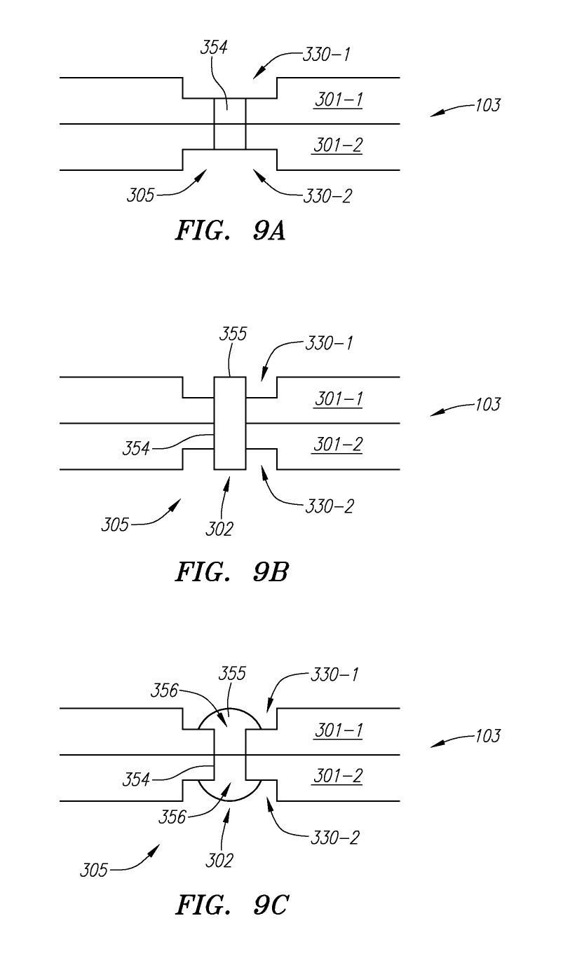

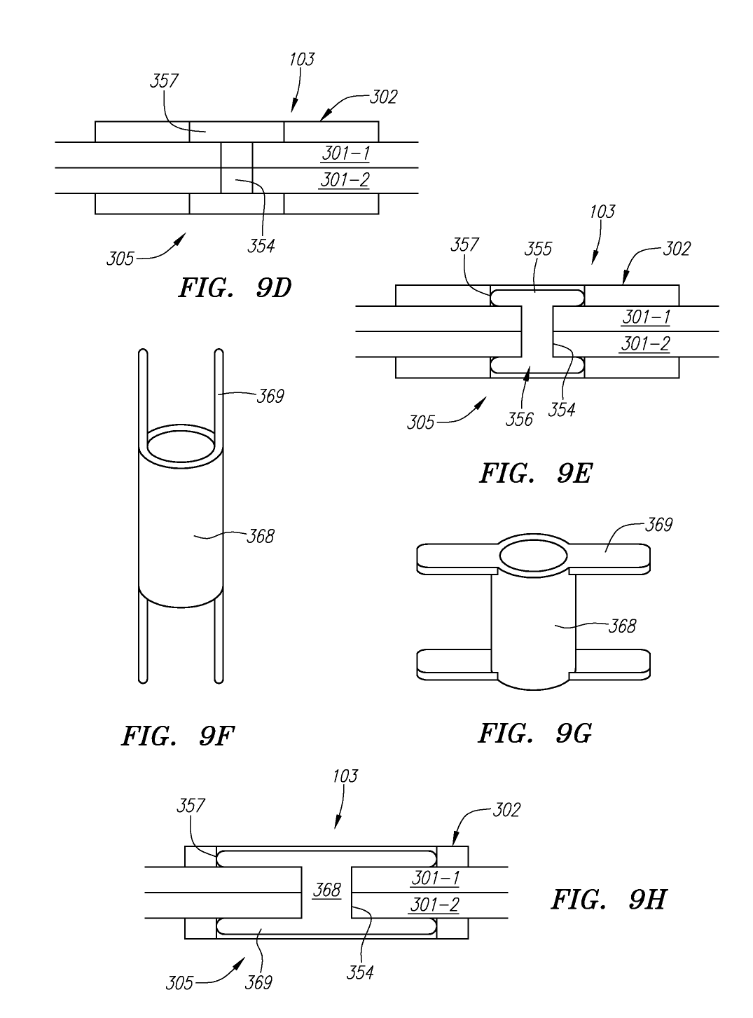

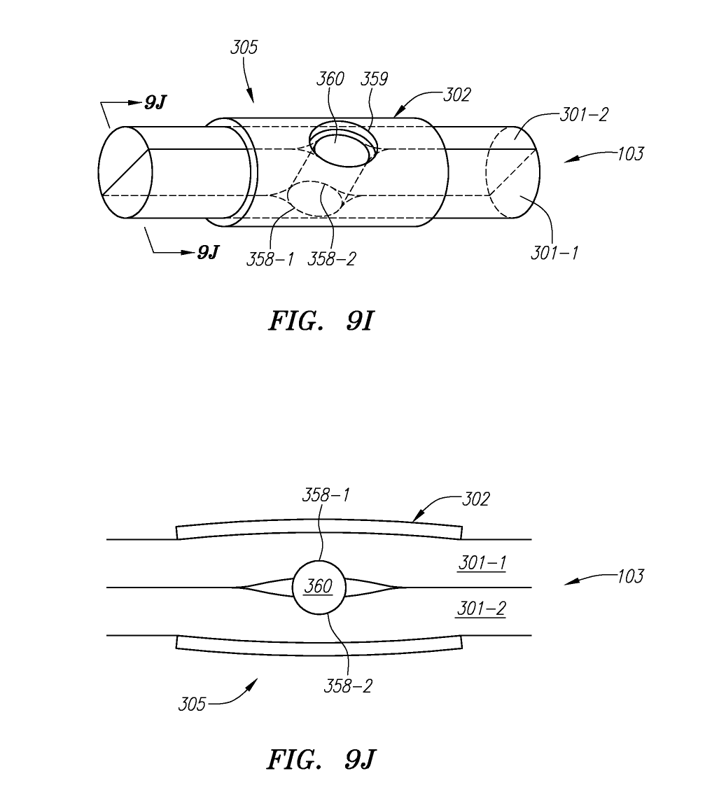

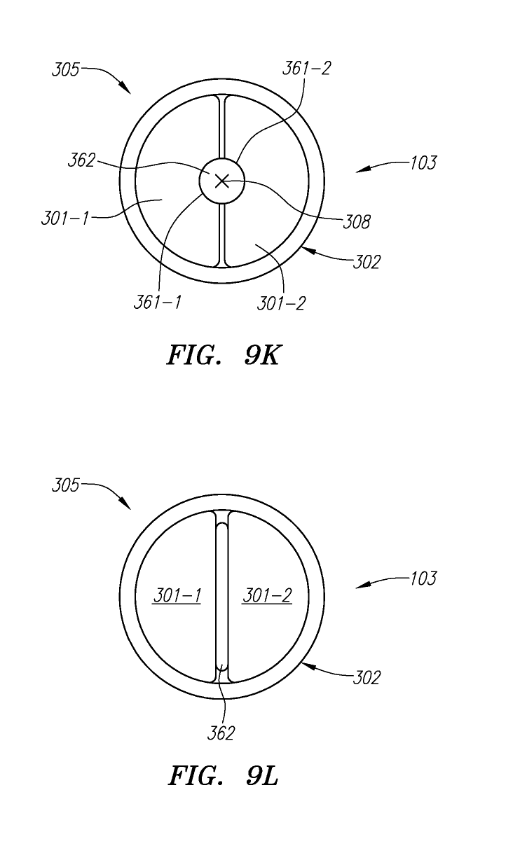

FIGS. 9A-C are cross-sectional views depicting an exemplary embodiment of clip 103 where the coupling device is configured as a rivet. FIG. 9A is a cross-sectional view along the center axis of the central portion 305 of clip 103 showing wires 301 located adjacent to each other without the presence of coupling device 302. An aperture 354 configured to receive a rivet-like member, is located in the recessed portions 330 of wires 301.

FIG. 9B shows rivet-like member 355 after being advanced through aperture 354. Here, rivet-like member 355 is generally cylindrical and has a longer length than aperture 354. FIG. 9C depicts rivet-like member 355 after being formed into a configuration suitable for locking wires 301 together. In this embodiment, each end of rivet-like member 355 has been deformed, or pressed, into enlarged portion 356 to lock rivet-like member 355 into place between wires 301.

It should be noted that any number of rivets can be used as coupling devices 302 and their configuration can be varied from that as shown here. For instance, rivet-like member 355 can be configured to fit within an aperture 354 having a non-cylindrical profile. Rivet-like member 354 can be formed with one end already enlarged, or rivet-like member 355 can include two preformed pieces that can be entered into either side of aperture 354 and coupled together. It should also be noted that other coupling devices can be used, such as screws, pins or clips.

FIGS. 9D-E are cross-sectional views depicting central portion 305 of another exemplary embodiment of clip 103 with a rivet-like member 355. FIG. 9D depicts wires 301 adjacent to each other with an aperture 354 formed therein. Wires 301 are also covered by a tubular coupling device 302 having a relatively larger aperture 357 formed therein in a position corresponding to the position of aperture 354.

FIG. 9E depicts clip 103 with rivet-like member 355 located therein. Here, rivet-like member 355 has enlarged portions 356 that fit within aperture 357 of coupling device 302. This embodiment allows rivet-like member 355 to be easily used in conjunction with coupling device 302. Because of the presence of enlarged portion 356 within aperture 357, this embodiment also allows rivet-like member 355 to anchor coupling device 302 into place. Rivet-like member 355 and tubular coupling device 302 can together act to maintain wires 301-1 in locked relation to each other. Instead of using a rivet-like member to lock wires 301 together, coupling device 302 can be molded over wires 301, e.g., such as with an injection-molded polymer. The polymeric or other moldable material flows into aperture 357 and over wires 301 and, upon hardening, forms an integrally-locked coupling device 302. Also, instead of using a rivet-like member or a molded coupling device, a tubular member with deflectable tabs can be used such as that depicted in FIGS. 9F-G. FIGS. 9F-G are perspective views of a tubular body 368 having two deflectable tabs 369 on both ends, tabs 369 being shown in the undeflected and deflected configurations, respectively. Tabs 369 can be deflected such that they lie in the configuration of FIG. 9F (generally parallel to the center axis of tubular body 368) to allow the tubular body 368 to be advanced through wire apertures 354. Once in place, tabs 369 can be deflected (or are biased to self-deflect) into the configuration depicted in FIG. 9G (generally perpendicular to the center axis of tubular body 368), where tabs 369 are received in coupling device apertures 357, as depicted in the cross-sectional view of FIG. 9H.

FIGS. 9I-J depict another exemplary embodiment of clip 103. As shown in the perspective view of FIG. 9I, grooves 358-1 and 358-2 are formed across both wires 301-1 and 301-2, respectively (surfaces edges that are obscured are denoted with dashed lines). Grooves 358 align to form an aperture extending across the width of wires 301-1 and 301-2. Coupling device 302 is shown in position over wires 301. Coupling device 302 has an aperture 359 which is preferably aligned over grooves 358-1 and 358-2.

FIG. 9J is a longitudinal cross-sectional view taken along line 9J-9J of FIG. 9I. In this cross-sectional view a wedge, or shim, 360 is shown after being lodged within grooves 358-1 and 358-2. This wedge applies pressure forcing wires 301-1 and 301-2 away from each other and against the wall of coupling device 302 to create a tighter and more stable fit. It should be noted that although wedge 360 is shown to be generally cylindrical in FIG. 9J, any shape wedge can be used that will act to force wires 301 apart. Preferably, clip 103 is configured to retain wedge 360 within grooves 358 without additional means, however wedge 360 can be sealed in place by rotating coupling device 302 such that apertures 359 (shown in FIG. 9IF) are no longer aligned with grooves 358, or by the use of adhesive, welding and/or soldering and the like.

FIGS. 9K-L are cross-sectional end views depicting the central portion 305 of exemplary embodiments of clip 103. In FIG. 9K, wires 301-1 and 301-2 include grooves 361-1 and 361-2, respectively, located longitudinally along center axis 308 of clip 103. Like the embodiment described with respect to FIGS. 9I-J, a wedge 362 is used to act to force wires 301-1 and 301-2 apart within coupling device 302. Again, this creates a tighter and more stable fit of wires 301 within coupling device 302. Wedge 362 is aligned with coupling device 302 so that they both reside in generally the same region of clip 103.

FIG. 9L is a cross-sectional end view showing wires 301 within coupling device 302. Here, a sheet-like, or ribbon-like wedge 362 is placed between wires 301-1 and 301-2. Although possible, in this embodiment, no additional groove(s) to receive wedge 362 are used. Based on this description herein, one of skill in the art will readily recognize the many different permissible shapes and configurations for wedge 362 that will act to force wires 301 apart.

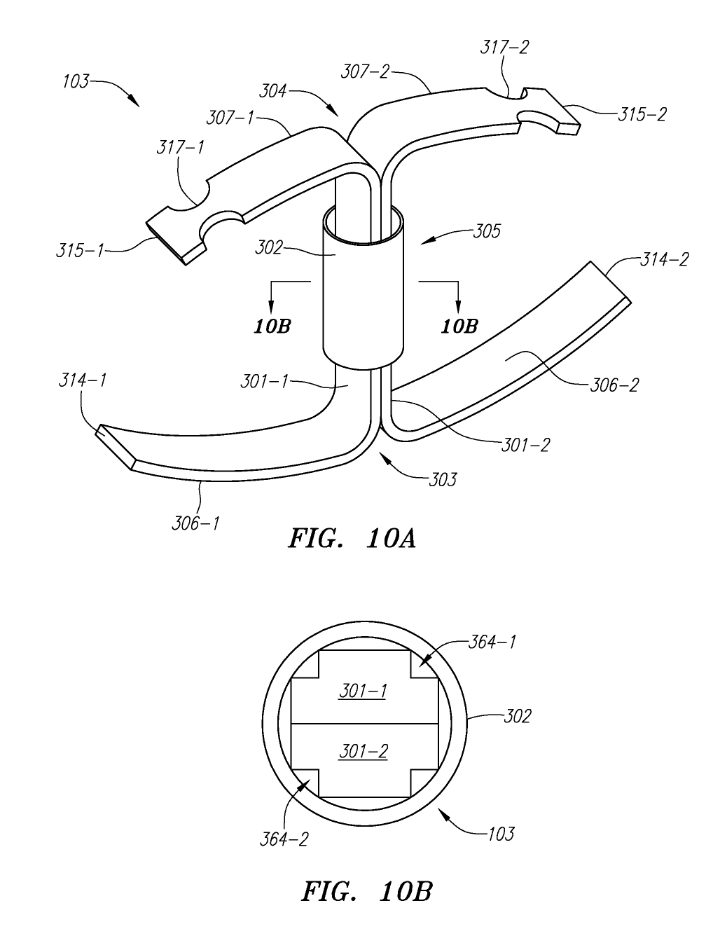

Turning now to the configuration of wires 301, FIGS. 10A-B depict an exemplary embodiment of clip 103 in the at-rest state where wires 301-1 and 301-2 have a rectangular cross-sectional profile. Wires 301 can be fabricated in any manner from any desired form of material, such as sectioned ribbon-like wire stock or etched/cut from a planar sheet of material. It should be noted that LA members 306 and RA members 307 can cross, similar to the embodiment described with respect to FIG. 2A, even though they are not shown to in FIG. 10A.

FIG. 10B is a cross-sectional view of clip 103 taken along line 10B-10B of FIG. 10A (for ease of illustration, LA members 306 are not shown) depicting wires 301-1 and 301-2 within coupling device 302. If desired, because coupling device 302 is cylindrical in this embodiment, wires 301 can each include a stepped portion 364 that reduces the cross-sectional profile of wires 301 so that they more efficiently fill the space within coupling device 302. Any number of step portions can be used and these stepped portions can be present along the longitudinal length of wires 301 corresponding to the length of coupling device 302. It should be noted that any cross-sectional shape of coupling device 302 can be used, including rectangular and other non-circular shapes in order to adequately engage rectangular wires 301.

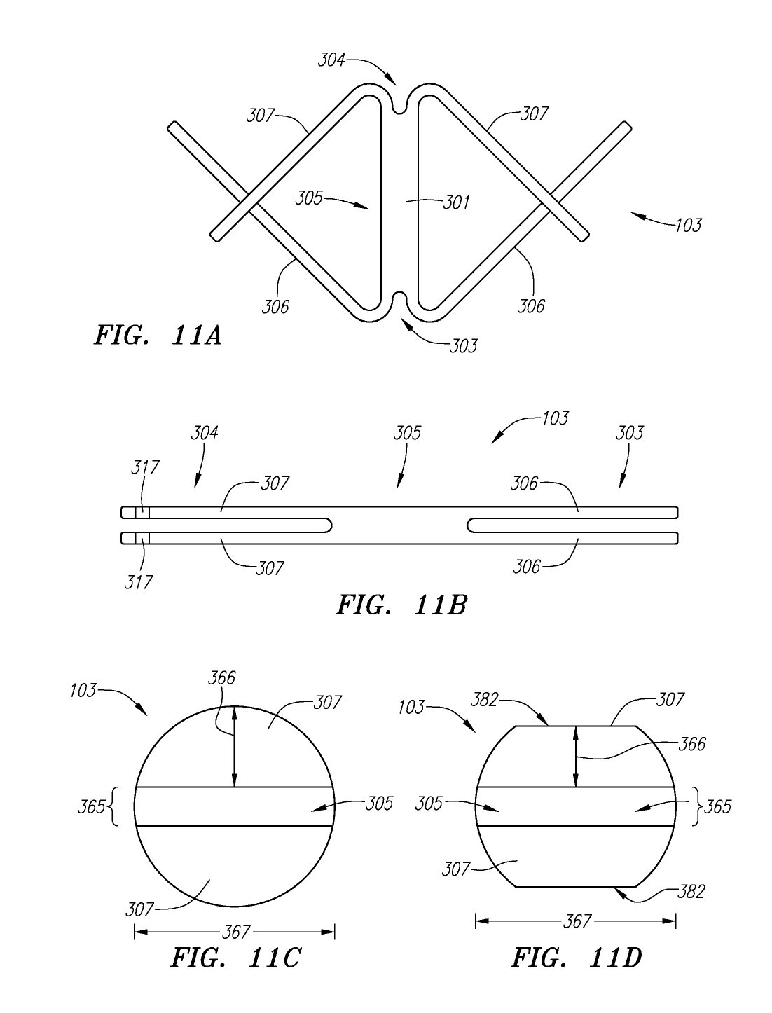

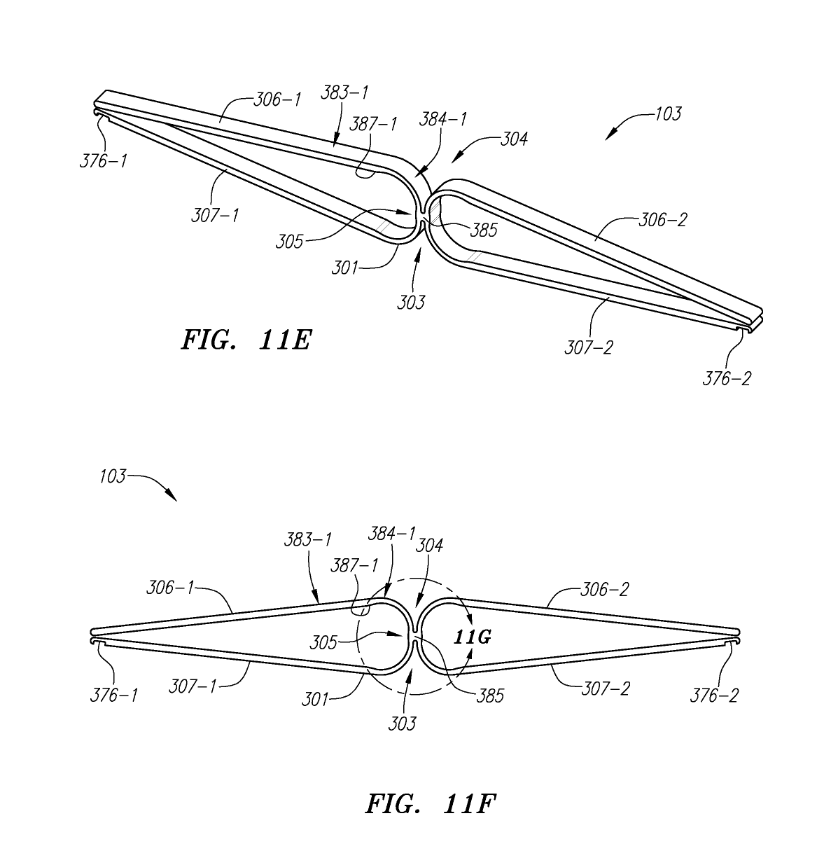

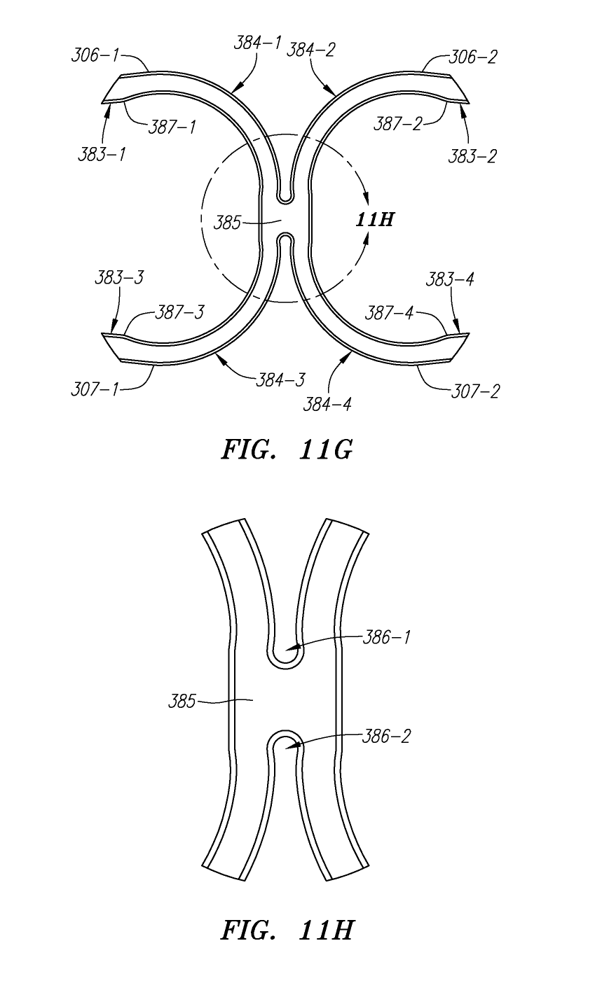

FIGS. 11A-D depict exemplary embodiments of clip 103 formed initially from a single piece of material. FIG. 11A is a side view of an exemplary embodiment of clip 103 having a wire-like body 301 with a monolithic core. Although coatings or other materials, e.g., radiopaque markers, can be added, this monolithic core construction can provide a relatively high resistance to stress and loads while implanted within the septal wall, allows the width and thickness of LA/RA members 306/307 to be easily varied and is relatively easy to manufacture, as compared to multi-component devices or devices with a tubular central section. Solid (i.e., continuous or without a seam/gap) central portion 305 eliminates the need for a coupling device 302 and simplifies the fabrication and construction of clip 103. The absence of coupling device 302 further allows clip 103 to maintain a relatively more uniform cross-sectional profile which allows for a more efficient housing within the delivery needle and/or catheter and also reduces the size of the manmade opening through the septal wall.

FIG. 11B depicts clip 103 in the relatively straightened configuration. FIGS. 11C-D are end views depicting exemplary embodiments of clip 103 while in the relatively straight configuration. In the embodiment of FIG. 11C, RA members 307 and LA members 306 (not shown) have a generally D-shaped cross-sectional profile. Gap 365 is shown separating adjacent RA members 307. This gap 365 can be present on either end of clip 103 and can be varied as desired in relation to the thickness of the adjacent members 306 and/or 307. For instance, when RA members 307 are relatively thick, these members provide increased closure force but also have increased resistance to deflection, whereas a relatively thinner member will provide relatively less closure force but will be more readily deflectable.

In the embodiment of FIG. 11D, the outer sides of each RA member 307 and LA member 306 (not shown) have a flat surface 382. This flattened outer surface 382, in conjunction with the relatively flat inner surfaces, gives the members 306 and 307 a relatively overall flat profile. Here, the maximum thickness 366 is reduced while width 367 remains constant, as compared to the embodiment described with respect to FIG. 11C having the same size gap 365. This can allow RA members 307 to more readily deflect.

The embodiments described with respect to FIGS. 11A-D can be fabricated in any desired manner and from any form of material. For instance, clip 103 can be sectioned from generally cylindrical wire stock, the ends of which can be split to form LA/RA members 306/307 as well as gap 365. Splitting of the ends can be accomplished in any desired manner, including but not limited to the use of machine cutting tools, laser or thermal cutting, chemical etching, any combination thereof and the like. Any flattened surface can be provided on the initial stock or added by grinding, etching, pressing and the like.

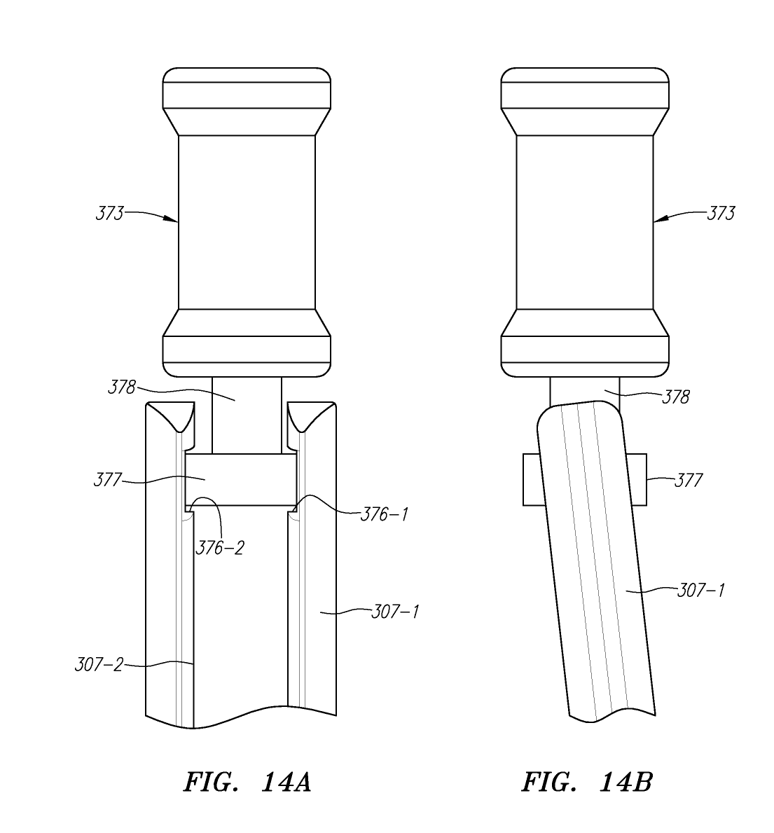

FIGS. 11E-H depict another exemplary embodiment of clip 103 with a monolithic, or unibody, core construction (similar to that described with respect to FIGS. 11A-D), where each LA member 306 and each RA member 307 meet at a central connection (or band) 385. FIG. 11E is a perspective view and FIG. 11F is a side view of clip 103 in the at-rest configuration. In this embodiment, LA members 306 and RA members 307 each have the same length and, although members 306 and 307 do not cross-over like the embodiments of FIGS. 2A-C, 2F, 4A and 11A, they can readily be configured to cross-over to provide added closure force in a manner similar to that described with respect to those embodiments. Each RA member 307 has a recessed portion 376 for interfacing with the delivery device and will be described in more detail with respect to FIGS. 14A-B.

To facilitate deflection of clip 103 from the relatively straightened configuration towards the at-rest configuration shown here, and also to provide increased stiffness along the length of each member to achieve a higher degree of closure, each member 306 and 307 has a relatively straight and thick portion 383 adjacent to a relatively curved and thin portion 384 that, in turn is adjacent to the central connection 385. This is shown in greater detail in FIG. 11G, which is an enlarged depiction of region 11G of FIG. 11F. A gradual transition 387 between portions 383 and 384 is present on the inner surface of each member at a position where the curved and straight portions meet, as shown here in the at-rest configuration. FIG. 11H is an enlarged depiction of region 11H of FIG. 11G. This depiction shows the presence of keyholes 386-1 and 386-2 at the interface between each LA member 306 and each RA member 307. Keyholes 386 are a variation in the profile of the clip for stress/strain relief. Here, keyholes 386 are rounded features that have a lateral dimension that is wider than the spacing of the immediately adjacent members. Viewed from the side perspective of FIG. 11H, keyholes 386 have a semi-circular profile (or are a semi-circular channel) with a diameter that is greater than the spacing between the immediately adjacent members. Keyholes 386 provide strain relief when the clip is in the relatively straight configuration (e.g., FIG. 11D) for housing within the delivery device. Other feature shapes for stress relief can also be used.

The closure force of the clip 103 can be varied according to the clip's dimensions. The width of clip 103 (i.e., the dimension along the normal axis to FIG. 11F, which in this embodiment is the same as the length of the channel) can vary from about 0.010 inches, e.g., for neurovascular applications, treatment of aneurysms, and the like, to about 0.050 inches for treatment of PFO's, PDA's, and the like. These and even larger dimensions can be used in abdominal applications such as hernia treatments, gastrointestinal treatments, fundoplication, and the like. The closure force of the clip can also be varied according to the radius of curvature (A) of each member, as depicted in FIG. 11G. The thickness in relatively thick portion 383, and moreso in relatively thin portion 384 (B), can also increase closure force, as well as the length (C) of the relatively thin portion 384.

The embodiment described with respect to FIGS. 11E-G can be fabricated in any desired manner and from any form of material. For instance, clip 103 can be laser cut from a sheet of nitinol. The rough clip 103 can then be deburred, such as with a tumble process, to remove the excess nitinol from the clip edges. A polish (chemical or electrical) can then be performed followed by a passivation step. Passivation is preferred to strip off excess oxide and reform it into a minimal uniform thickness. A uniform oxide layer of minimal thickness reduces the risk of microcrack propagation and fatigue failure, and can have less nickel elution, improved biocompatibility and improved corrosion resistance.

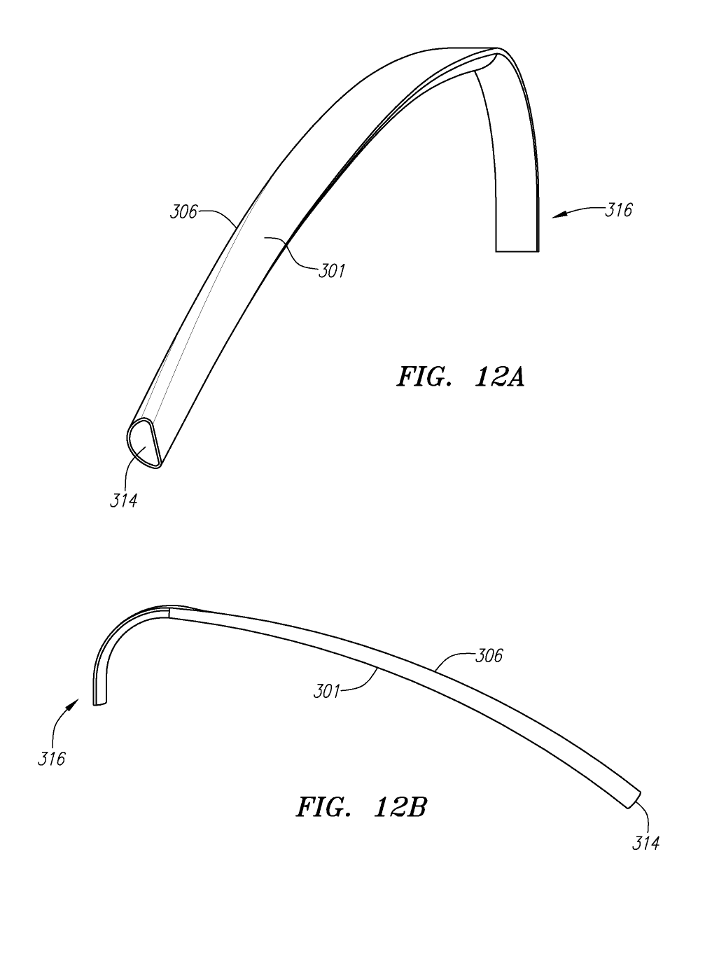

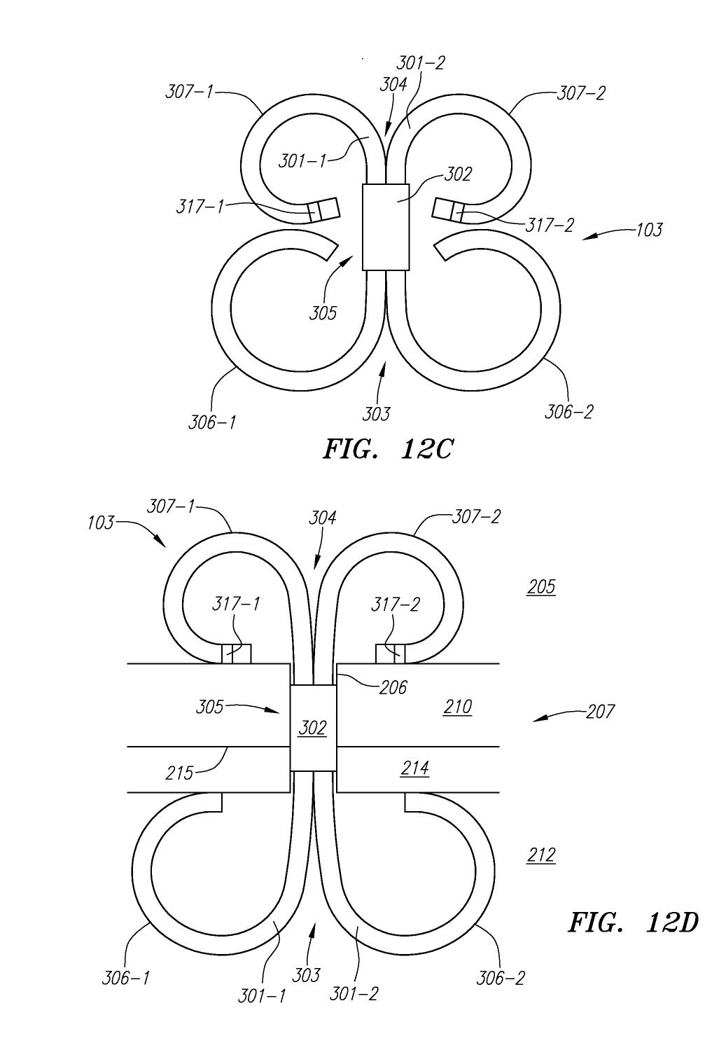

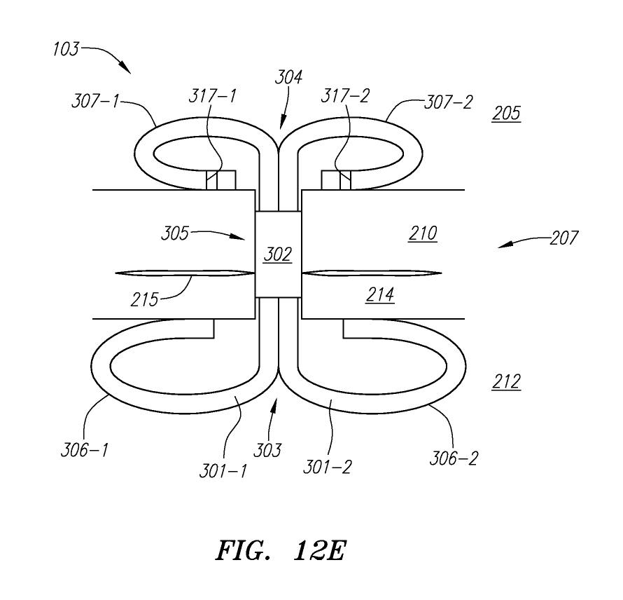

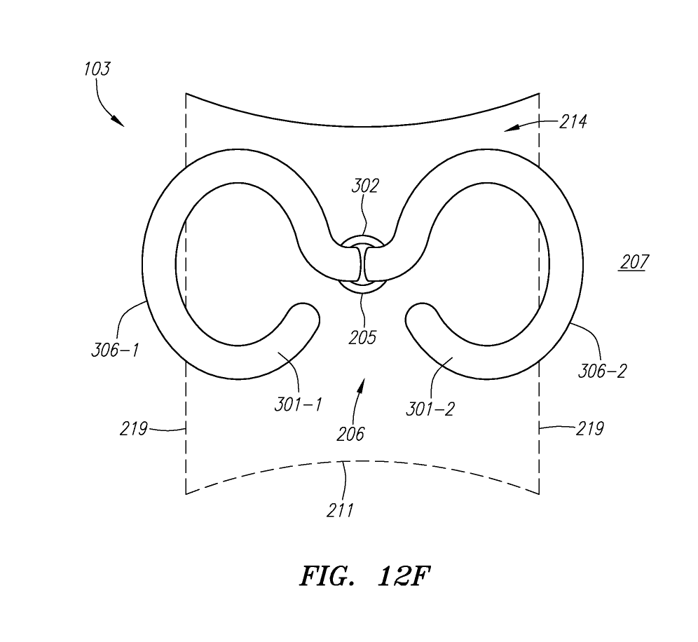

FIGS. 12A-B are perspective views, taken from different orientations, depicting an exemplary embodiment of LA member 306 having a twisted configuration. This configuration can be used with any of the embodiments described herein and can also be used with any or all of the LA or RA members. The cross-sectional profile of LA member 306 at the base portion 316 is rotated approximately 90 degrees between this base portion 316 and the end tip 314. Preferably, this rotation occurs continuously along the length of LA member 306 to minimize induced stress. This rotation can provide an increased moment of inertia at end tip 314 allowing LA member 306 to apply a greater closure force. It should be noted that although in this embodiment LA member 306 is rotated approximately 90 degrees, any amount of rotation can be applied. For instance, rotations of 15, 30, 45, 60 or 75 degrees would each allow LA member 306 to apply increasingly greater closure force at end tip 314.

FIGS. 12C-D are side views depicting another exemplary embodiment of clip 103. Here, LA/RA members 306/307 are looped to increase the closure force that can be applied to the septal tissue. FIG. 12C shows clip 103 in the at-rest state, while FIG. 12D shows clip 103 implanted within septal wall 207. Looped LA members 306 have a relatively larger radius of curvature than looped RA members 307 to allow a greater amount of septal tissue to be engaged. It should be noted that looped LA/RA members 306/307 can be configured with a relatively constant radius of curvature such as that shown, or the radius can be varied to provide, for instance, a more elliptical or flattened profile such as that depicted in FIG. 12E. It should be noted that this configuration can be implemented with any other exemplary embodiments described herein.

FIG. 12F is a left atrial view depicting a similar embodiment where the looped LA members 306 are biased to lay at least mostly flat on the septum primum 214. This looped (or annular) lay-flat configuration of members 306 allows increased coverage over the primum, which can increase the effectiveness of the PFO closure. Preferably, LA members 306 overlap the sidewalls 219 of the PFO to cover the entire width of the PFO tunnel. RA members 307 (not shown) can have a similar configuration, or can be relatively straight (such as that shown in FIG. 2B) or can have an upright looped configuration (such as that shown in FIGS. 12C-E) or any other desired configuration.

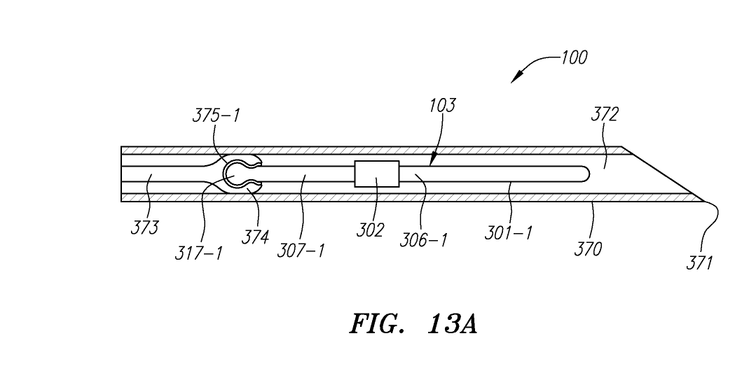

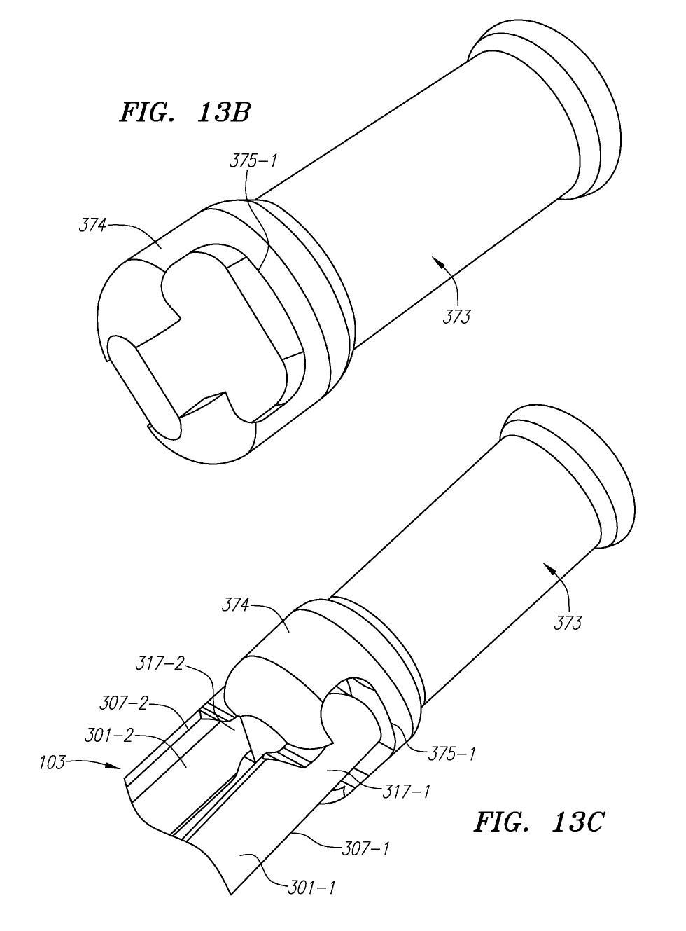

Turning now to delivery of clip 103, FIGS. 13A-C depict exemplary embodiments of portions of a delivery system 100 configured for intravascular delivery of clip 103. FIG. 13A is a partial cross-sectional view of needle-like member 370 having a substantially sharp, open distal end 371 configured to pierce septal tissue and an inner lumen 372 configured to house clip 103 and pusher 373. The proximal portion of one or both of RA members 307-1 and RA member 307-2 (not shown) can each include a relatively narrow neck region 317-1 located distal to the proximal end of RA member 307-1. Neck regions 317 are configured to allow engagement of clip 103 with pusher 373. Pusher 373, in this embodiment, includes a relatively wider distal portion 374 having recesses 375-1 and 375-2 (not shown) configured complementarily to the proximal portion of RA member 307-1 including neck 317-1.

The distal portion 374 of pusher 373 preferably has a slightly smaller width than the inner diameter of needle 370 so that a close fit is obtained and the needle walls maintain each RA member 307 within the corresponding recess 375 of pusher 373. This configuration allows pusher 373 to securely engage clip 103 and to both advance and retract clip 103 as desired.

FIGS. 13B-C are perspective views depicting the distal portion of pusher 373 in greater detail both with and without RA members 307, respectively. Based on the description herein, one of skill in the art will readily recognize the many various configurations of RA members 307 and recesses 375 that will allow pusher 373 to advance and retract clip 103.

FIGS. 14A-B are side views depicting an additional exemplary embodiment of pusher 373 coupled with the proximal portion of RA members 307-1 and 307-1. Here, RA members have recessed portions 376-1 and 376-2, which oppose each other when clip 103 is coupled with a disc-like retainer 377 positioned at the end of a strut 378 on pusher 373. Recessed portions 376 are in the outer surface of members 307 when in the at-rest state. Recessed portions 376 can have a stepped or rounded shape and are preferably large enough to allow some swivel with respect to pusher 373, which can facilitate delivery of the clip across a range of delivery angles that could be encountered during the procedure. The configuration depicted in FIGS. 14A-B allows for a high-degree of deployability in that there is little risk one or both of RA members will only occur after the interface has been advanced (and freed) from within the needle (not shown).

Any portion of clip 103 (e.g., wires 301 and/or coupling device 302, etc.) can be coated with any material as desired. Some exemplary coatings that can be used include coatings that are biodegradable, drug coatings (e.g., drugs can be released from hydrogels or polymer carriers where the polymer itself is a biodegradable material (e.g., poly(caprolactone), poly(D,L-lactic acid), polyorthoester, polyglycolides, polyanhydrides, erodable hydrogels and the like) or elastomers (e.g., polyurethane (PU), polydimethylsiloxane (PDMS) and the like), coatings that increase or decrease lubricity (e.g., hydrogels, polyurethane and the like), bioactive coatings (e.g., anti-platelet coatings, anti-microbial coatings and the like), coatings that inhibit thrombus formation or the occurrence an embolic events (e.g., heparin, pyrolytic carbon, phosphorylcholine and the like), and coatings that speed the healing response.

These coatings can be applied over the entire clip 103 or any portion thereof. Also, different portions of clip 103 can be coated with different coatings. For instance, because end portion 303 and LA members 306 lie within left atrium 212 in contact with the oxygenated arterial blood, it may be desirable to coat that region of clip 103 with a material designed to inhibit thrombus formation. On the other hand, end portion 304 and RA members 307 lie within right atrium 205 in contact with the oxygen-depleted venous blood, and it may therefore be desirable to coat that region of clip 103 with a material designed to accelerate or promote the healing response.

Clip 103 can also be coated in layers. For instance, in one exemplary embodiment clip 103 has two coatings applied: a first, underlying coating and a second coating situated over the first coating and exposed to the surrounding environment. The second, exposed coating can be a short term coating designed to dissolve over a desired time period. The second coating eventually dissolves enough to expose the underlying first coating, which can itself be configured to dissolve or can be a long term, permanent coating. Any number of coatings having any desired absorption rate or drug elution rate can be used.

Any portion of clip 103 can be made easier to view by an internal or external imaging device. For instance, in addition to the embodiments described with respect to FIGS. 2G-H, embodiment, radiopaque markings are added to LA/RA members 306/307 to make clip 103 viewable via fluoroscopy, while in another embodiment an echolucent coating is added to make clip 103 viewable with ultrasound devices. Clip 103 can be configured for use with any internal or external imaging device such as magnetic-resonance imaging (MRI) devices, computerized axial tomography (CAT) scan devices, X-ray devices, fluoroscopic devices, ultrasound devices and the like.

One should recognize that the various elements, features and configurations of clip, delivery system and method embodiments described in the incorporated U.S. Patent Application Publication number 2007/0129755 entitled “Clip-Based Systems and Methods for Treating Septal Defects” can each be likewise applied to the embodiments set forth herein. For instance, making reference to the figure numbers in the incorporated '755 publication, elements and/or features of: the various embodiments of LA/RA members 306/307 described with respect to FIGS. 7A-17J, the various embodiments of clips, delivery systems and methods for implanting the clip described with respect to FIGS. 3A-6C and 27A-28B, the various embodiments of the clip body described with respect to FIGS. 18A-24D, and the various embodiments pertaining to clip retrieval or recapture described with respect to FIGS. 25A-26G, can each be combined with or substituted for corresponding elements and/or features of the embodiments described herein, or supplemented to the embodiments described herein. Any of the embodiments of clip 103 can also be configured with LA members having sharp (or substantially sharp) distal end tips to allow the clip itself to act as the septal tissue piercing device, eliminating the need for a separate needle. The embodiments can be configured as tissue-piercing clips similar to those described in U.S. Pat. No. 6,776,784, entitled “Clip Apparatus for Closing Septal Defects and Methods of Use,” and PCT International Application serial no. PCT/US09/44647, entitled “Tissue-Piercing Implants and Other Devices for Treating Septal Defects,” filed on May 20, 2009, both of which are fully incorporated herein.

The devices, systems and methods described herein may be used in any part of the body, in order to treat a variety of disease states. Of particular interest are applications within hollow organs including but not limited to the heart and blood vessels (arterial and venous), lungs and air passageways, digestive organs (esophagus, stomach, intestines, biliary tree, etc.). The devices and methods will also find use within the genitourinary tract in such areas as the bladder, urethra, ureters, and other areas.

Other locations in which and around which the subject devices and methods find use include the liver, spleen, pancreas and kidney. Any thoracic, abdominal, pelvic, or intravascular location falls within the scope of this description.

The devices and methods may also be used in any region of the body in which it is desirable to appose tissues. This may be useful for causing apposition of the skin or its layers (dermis, epidermis, etc), fascia, muscle, peritoneum, and the like. For example, the subject devices may be used after laparoscopic and/or thoracoscopic procedures to close trocar defects, thus minimizing the likelihood of subsequent hernias. Alternatively, devices that can be used to tighten or lock sutures may find use in various laparoscopic or thoracoscopic procedures where knot tying is required, such as bariatric procedures (gastric bypass and the like) and Nissen fundoplication. The subject devices and methods may also be used to close vascular access sites (either percutaneous, or cut-down). These examples are not meant to be limiting.

The devices and methods can also be used to apply various patch-like or non-patchlike implants (including but not limited to Dacron, Marlex, surgical meshes, and other synthetic and non-synthetic materials) to desired locations. For example, the subject devices may be used to apply mesh to facilitate closure of hernias during open, minimally invasive, laparoscopic, and preperitoneal surgical hernia repairs.

It should be noted that various embodiments are described herein with reference to one or more numerical values. These numerical value(s) are intended as examples only and in no way should be construed as limiting the subject matter recited in any claim, absent express recitation of a numerical value in that claim.

While the embodiments are susceptible to various modifications and alternative forms, specific examples thereof have been shown in the drawings and are herein described in detail. It should be understood, however, that these embodiments are not to be limited to the particular form disclosed, but to the contrary, these embodiments are to cover all modifications, equivalents, and alternatives falling within the spirit of the disclosure.

What is claimed is:

1. An implantable device, comprising:
a first end portion comprising a first deflectable member and a second deflectable member, each deflectable member having an atraumatic end tip;
a second end portion comprising a first deflectable member and a second deflectable member, each deflectable member having an atraumatic end tip;
a solid central section between the first and second end portions, wherein the implantable device has a monolithic core where each of the deflectable members and solid central section are continuous, and is configured for implantation within a man-made opening in a septal wall, the implantable device being biased to deflect between an elongate state and an at-rest state,
wherein the first and second deflectable members of the first end portion are adjacent to each other in the elongate state and extend away from each other by a greater amount in the at-rest state, and the first and second deflectable members of the second end portion are adjacent each other in the elongate state and extend away from each other by a greater amount in the at-rest state.

2. The implantable device of claim 1, further comprising a keyhole located at the interface between the first and second deflectable members of the first end portion.

3. The implantable device of claim 2, wherein each member has a first and a second portion, the second portion being located between the first portion and the solid central section, wherein the first portion is and relatively thick and the second portion is relatively thin.

4. The implantable device of claim 3, wherein the second portion is curved in the at-rest state.

5. The implantable device of claim 4, wherein the second portion is relatively straight in the elongate state.

6. The implantable device of claim 4, wherein the first portion is relatively straight in the at-rest and elongate states.

7. The implantable device of claim 3, wherein a gradual transition is present between the first and second portions of each deflectable member.

8. The implantable device of claim 7, wherein, in the at-rest state, the gradual transition is located between the first portion in a relatively straight state and the second portion in a relatively curved state.

9. The implantable device of claim 8, wherein the gradual transitions are located on the inner surface of the deflectable members of the implantable device in the at-rest state.

10. The implantable device of claim 1, wherein the first and second deflectable members of the first end portion each have a recessed portion on the outer surface of the deflectable members in the at-rest state.

11. The implantable device of claim 1, wherein the first end portion comprises only two deflectable members and the second end portion comprises only two deflectable members.

12. The implantable device of claim 11, wherein, in the at-rest state, the first deflectable member of the first end portion crosses the first deflectable member of the second end portion and the second deflectable member of the first end portion crosses the second deflectable member of the second end portion.

13. The implantable device of claim 11, wherein, in the at-rest state, the first deflectable member of the first end portion does not cross the first deflectable member of the second end portion and the second deflectable member of the first end portion does not cross the second deflectable member of the second end portion, the first deflectable members being located on one side of the implantable device and the second deflectable members being located on the opposite side.

14. The implantable device of claim 1, further comprising a keyhole located at the interface between the first and second deflectable members of the first end portion, the keyhole being a semi-circular channel.

15. The implantable device of claim 1, wherein the length of the channel is greater than the thickness of the adjacent deflectable members.
